SEQUENCE LISTING (18 sequences)

SEQ ID NO: 1 - DNA, 1140 bp, Bacillus licheniformis
  sig_peptide (1)..(87)
  CDS (1)..(1137)
  misc_structure (88)..(315): Proregion
atgatgaggaaaaagagtttttggcttgggatgctgacggccttcatgctcgtgttcacgatggcattcagcgattccgcttctgctgctcaaccggcgaaaaatgttgaaaaggattatattgtcggatttaagtcaggagtgaaaaccgcatctgtcaaaaaggacatcatcaaagagagcggcggaaaagtggacaagcagtttagaatcatcaacgcggcaaaagcgaagctagacaaagaagcgcttaaggaagtcaaaaatgatccggatgtcgcttatgtggaagaggatcatgtggcccatgccttggcgcaaaccgttccttacggcattcctctcattaaagcggacaaagtgcaggctcaaggctttaagggagcgaatgtaaaagtagccgtcctggatacaggaatccaagcttctcatccggacttgaacgtagtcggcggagcaagctttgtggctggcgaagcttataacaccgacggcaacggacacggcacacatgttgccggtacagtagctgcgcttgacaatacaacgggtgtattaggcgttgcgccaagcgtatccttgtacgcggttaaagtactgaattcaagcggaagcggatcatacagcggcattgtaagcggaatcgagtgggcgacaacaaacggcatggatgttatcaatatgagccttgggggagcatcaggctcgacagcgatgaaacaggcagtcgacaatgcatatgcaagaggggttgtcgttgtagctgcagcagggaacagcggatcttcaggaaacacgaatacaattggctatcctgcgaaatacgattctgtcatcgctgttggtgcggtagactctaacagcaacagagcttcattttccagcgtcggagcagagcttgaagtcatggctcctggcgcaggcgtgtacagcacttacccaacgaacacttatgcaacattgaacggaacgtcaatggcttctcctcatgtagcgggagcagcagctttgatcttgtcaaaacatccgaacctttcagcttcacaagtccgcaaccgtctctccagcacggcgacttatttgggaagctccttctactatgggaaaggtctgatcaatgtcgaagctgccgctcaataa

SEQ ID NO: 2 - protein, 379 aa, Bacillus licheniformis
MMRKKSFWLGMLTAFMLVFTMAFSDSASAAQPAKNVEKDYIVGFKSGVKTASVKKDIIKESGGKVDKQFRIINAAKAKLDKEALKEVKNDPDVAYVEEDHVAHALAQTVPYGIPLIKADKVQAQGFKGANVKVAVLDTGIQASHPDLNVVGGASFVAGEAYNTDGNGHGTHVAGTVAALDNTTGVLGVAPSVSLYAVKVLNSSGSGSYSGIVSGIEWATTNGMDVINMSLGGASGSTAMKQAVDNAYARGVVVVAAAGNSGSSGNTNTIGYPAKYDSVIAVGAVDSNSNRASFSSVGAELEVMAPGAGVYSTYPTNTYATLNGTSMASPHVAGAAALILSKHPNLSASQVRNRLSSTATYLGSSFYYGKGLINVEAAAQ

SEQ ID NO: 3 - DNA, 948 bp, Bacillus licheniformis
  CDS (1)..(948)
  sig_peptide (1)..(93)
  misc_structure (94)..(282): Pro-peptide
ttggttagtaaaaagagtgttaaacgaggtttgatcacaggtctcattggtatttctatttattctttaggtatgcacccggcccaagccgcgccatcgcctcatactcctgtttcaagcgatccttcatacaaagcggaaacatcggttacttatgacccacacattaagagcgatcaatacggcttgtattcaaaagcgtttacaggcaccggcaaagtgaatgaaacaaaggaaaaagcggaaaaaaagtcacccgccaaagctccttacagcattaaatcggtgattggttctgatgatcggacaagggtcaccaacacaaccgcatatccgtacagagcgatcgttcatatttcaagcagcatcggttcatgcaccggatggatgatcggtccgaaaaccgtcgcaacagccggacactgcatctatgacacatcaagcggttcatttgccggtacagccactgtttcgccgggacggaacgggacaagctatccttacggctcagttaaatcgacgcgctactttattccgtcaggatggagaagcggaaacaccaattacgattacggagcaatcgaactaagcgaaccgatcggcaatactgtcggatacttcggatactcgtacactacttcatcacttgttgggacaactgttaccatcagcggctacccaggcgataaaacagcaggcacacaatggcagcattcaggaccgattgccatctccgaaacgtataaattgcagtacgcaatggacacgtacggaggacaaagcggttcaccggtattcgaacaaagcagctccagaacgaactgtagcggtccgtgctcgcttgccgtacacacaaatggagtatacggcggctcctcgtacaacagaggcacccggattacaaaagaggtgttcgacaatttgaccaactggaaaaacagcgcacaa

SEQ ID NO: 4 - protein, 316 aa, Bacillus licheniformis
LVSKKSVKRGLITGLIGISIYSLGMHPAQAAPSPHTPVSSDPSYKAETSVTYDPHIKSDQYGLYSKAFTGTGKVNETKEKAEKKSPAKAPYSIKSVIGSDDRTRVTNTTAYPYRAIVHISSSIGSCTGWMIGPKTVATAGHCIYDTSSGSFAGTATVSPGRNGTSYPYGSVKSTRYFIPSGWRSGNTNYDYGAIELSEPIGNTVGYFGYSYTTSSLVGTTVTISGYPGDKTAGTQWQHSGPIAISETYKLQYAMDTYGGQSGSPVFEQSSSRTNCSGPCSLAVHTNGVYGGSSYNRGTRITKEVFDNLTNWKNSAQ

SEQ ID NO: 5 - protein, 379 aa, Bacillus licheniformis
  SIGNAL (1)..(29)
  PROPEP (30)..(105)
  mat_peptide (106)..(379)
MMRKKSFWLGMLTAFMLVFTMAFSDSASAAQPAKNVEKDYIVGFKSGVKTASVKKDIIKESGGKVDKQFRIINAAKAKLDKEALKEVKNDPDVAYVEEDHVAHALAQTVPYGIPLIKADKVQAQGFKGANVKVAVLDTGIQASHPDLNVVGGASFVAGEAYNTDGNGHGTHVAGTVAALDNTTGVLGVAPSVSLYAVKVLNSSGSGTYSGIVSGIEWATTNGMDVINMSLGGPSGSTAMKQAVDNAYARGVVVVAAAGNSGSSGNTNTIGYPAKYDSVIAVGAVDSNSNRASFSSVGAELEVMAPGAGVYSTYPTSTYATLNGTSMASPHVAGAAALILSKHPNLSASQVRNRLSSTATYLGSSFYYGKGLINVEAAAQ

SEQ ID NO: 6 - protein, 381 aa, Bacillus subtilis var. natto
  SIGNAL (1)..(23)
  PROPEP (24)..(106)
  mat_peptide (107)..(381)
MRSKKLWISLLFALTLIFTMAFSNMSAQAAGKSSTEKKYIVGFKQTMSAMSSAKKKDVISEKGGKVQKQFKYVNAAAATLDEKAVKELKKDPSVAYVEEDHIAHEYAQSVPYGISQIKAPALHSQGYTGSNVKVAVIDSGIDSSHPDLNVRGGASFVPSETNPYQDGSSHGTHVAGTIAALNNSIGVLGVAPSASLYAVKVLDSTGSGQYSWIINGIEWAISNNMDVINMSLGGPTGSTALKTVVDKAVSSGIVVAAAAGNEGSSGSTSTVGYPAKYPSTIAVGAVNSSNQRASFSSVGSELDVMAPGVSIQSTLPGGTYGAYNGTSMATPHVAGAAALILSKHPTWTNAQVRDRLESTATYLGNSFYYGKGLINVQAAAQ

SEQ ID NO: 7 - protein, 275 aa, Bacillus pumilus (mesentericus)
  mat_peptide (1)..(275)
AQSVPYGISQIKAPALHSQGYTGSNVKVAVIDSGIDSSHPDLNVRGGASFVPSETNPYQDGSSHGTHVAGTIAALNNSIGVLGVAPSSALYAVKVLDSTGSGQYSWIINGIEWAISNNMDVINMSLGGPTGSTALKTVVDKAVSSGIVVAAAAGNEGSSGSTSTVGYPAKYPSTIAVGAVNSANQRASFSSAGSELDVMAPGVSIQSTLPGGTYGAYNGTSMATPHVAGAAALILSKHPTWTNAQVRDRLESTATYLGSSFYYGKGLINVQAAAQ

SEQ ID NO: 8 - protein, 381 aa, Bacillus subtilis
  SIGNAL (1)..(23)
  PROPEP (24)..(106)
  mat_peptide (107)..(381)
MRSKKLWISLLFALTLIFTMAFSNMSVQAAGKSSTEKKYIVGFKQTMSAMSSAKKKDVISEKGGKVQKQFKYVNAAAATLDEKAVKELKKDPSVAYVEEDHIAHEYAQSVPYGISQIKAPALHSQGYTGSNVKVAVIDSGIDSSHPDLNVRGGASFVPSETNPYQDGSSHGTHVAGTIAALNNSIGVLGVSPSASLYAVKVLDSTGSGQYSWIINGIEWAISNNMDVINMSLGGPTGSTALKTVVDKAVSSGIVVAAAAGNEGSSGSTSTVGYPAKYPSTIAVGAVNSSNQRASFSSAGSELDVMAPGVSIQSTLPGGTYGAYNGTSMATPHVAGAAALILSKHPTWTNAQVRDRLESTATYLGNSFYYGKGLINVQAAAQ

SEQ ID NO: 9 - protein, 381 aa, Bacillus stearothermophilus
  SIGNAL (1)..(29)
  PROPEP (30)..(106)
  mat_peptide (107)..(381)
MRSKKLWISLLFALTLIFTMAFSNMSVQAAGKSSTEKKYIVGFKQTMSAMSSAKKKDVISEKGGKVQKQFKYVNAAAATLDEKAVKELKKDPSVAYVEEDHIAHEYAQSVPYGISQIKAPALHSQGYTGSNVKVAVIDSGIDSSHPDLNVRGGASFVPSETNPYQDGSSHGTHVAGTIAALNNSIGVLGVSPSASLYAVKVLDSTGSGQYSWIINGIEWAISNNMDVINMSLGGPSGSTALKTVVDKAVSSGIVVAAAAGNEGSSGSSSTVGYPAKYPSTIAVGAVNSSNQRASFSSAGSELDVMAPGVSIQSTLPGGTYGAYNGTSMATPHVAGAAALILSKHPTWTNAQVRDRLESTATYLGNSFYYGKGLINVQAAAQ

SEQ ID NO: 10 - protein, 382 aa, Bacillus amyloliquefaciens
  SIGNAL (1)..(32)
  PROPEP (33)..(107)
  mat_peptide (108)..(382)
MRGKKVWISLLFALALIFTMAFGSTSSAQAAGKSNGEKKYIVGFKQTMSTMSAAKKKDVISEKGGKVQKQFKYVDAASATLNEKAVKELKKDPSVAYVEEDHVAHAYAQSVPYGVSQIKAPALHSQGYTGSNVKVAVIDSGIDSSHPDLKVAGGASMVPSETNPFQDNNSHGTHVAGTVAALNNSIGVLGVAPSASLYAVKVLGADGSGQYSWIINGIEWAIANNMDVINMSLGGPSGSAALKAAVDKAVASGVVVVAAAGNEGTSGSSSTVGYPGKYPSVIAVGAVDSSNQRASFSSVGPELDVMAPGVSIQSTLPGNKYGAYNGTSMASPHVAGAAALILSKHPNWTNTQVRSSLENTTTKLGDSFYYGKGLINVQAAAQ

SEQ ID NO: 11 - protein, 269 aa, Bacillus lentus
  mat_peptide (1)..(269)
AQSVPWGISRVQAPAAHNRGLTGSGVKVAVLDTGISTHPDLNIRGGASFVPGEPSTQDGNGHGTHVAGTIAALNNSIGVLGVAPSAELYAVKVLGASGSGSVSSIAQGLEWAGNNGMHVANLSLGSPSPSATLEQAVNSATSRGVLVVAASGNSGAGSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 12 - protein, 380 aa, Bacillus clausii
  SIGNAL (1)..(27)
  PROPEP (28)..(111)
  mat_peptide (112)..(380)
MKKPLGKIVASTALLISVAFSSSIASAAEEAKEKYLIGFNEQEAVSEFVEQVEANDEVAILSEEEEVEIELLHEFETIPVLSVELSPEDVDALELDPAISYIEEDAEVTTMAQSVPWGISRVQAPAAHNRGLTGSGVKVAVLDTGISTHPDLNIRGGASFVPGEPSTQDGNGHGTHVAGTIAALNNSIGVLGVAPSAELYAVKVLGASGSGSVSSIAQGLEWAGNNGMHVANLSLGSPSPSATLEQAVNSATSRGVLVVAASGNSGAGSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 13 - protein, 378 aa, Bacillus sp.
  SIGNAL (1)..(27)
  PROPEP (28)..(110)
  mat_peptide (111)..(378)
MNKKMGKIVAGTALIISVAFSSSIAQAAEEAKEKYLIGFKEQEVMSQFVDQIDGDEYSISSQAEDVEIDLLHEFDFIPVLSVELDPEDVDALELDPAIAYIEEDAEVTTMQTVPWGINRVQAPIAQSRGFTGTGVRVAVLDTGISNHADLRIRGGASFVPGEPNISDGNGHGTQVAGTIAALNNSIGVLGVAPNVDLYGVKVLGASGSGSISGIAQGLQWAANNGMHIANMSLGSSAGSATMEQAVNQATASGVLVVAASGNSGAGNVGFPARYANAMAVGATDQNNNRATFSQYGAGLDIVAPGVGVQSTVPGNGYASFNGTSMATPHVAGVAALVKQKNPSWSNVQIRNHLKNTATNLGNTTQFGSGLVNAEAATR

SEQ ID NO: 14 - protein, 269 aa, Thermomyces lanuginosus [sequence omitted]

SEQ ID NO: 15 - protein, 274 aa, Humicola lanuginosa
  VARIANT (1)..(269)
  mat_peptide (6)..(269)
SPIRREVSQDLFNQFNLFAQYSAAAYCGKNNDAPAGTNITCTGNACPEVEKADATFLYSFEDSGVGDVTGFLALDNTNKLIVLSFRGSRSIENWIGNLNFDLKEINDICSGCRGHDGFTSSWRSVADTLRQKVEDAVREHPDYRVVFTGHSLGGALATVAGADLRGNGYDIDVFSYGAPRVGNRAFAEFLTVQTGGTLYRITHTNDIVPRLPPREFGYSHSSPEYWIKSGTLVPVRRRDIVKIEGIDATGGNNQPNIPDIPAHLWYFGLIGTCL

SEQ ID NO: 16 - protein, 513 aa, Bacillus stearothermophilus
  VARIANT (1)..(513)
AAPFNGTMMQYFEWYLPDDGTLWTKVANEANNLSSLGITALWLPPAYKGTSRSDVGYGVYDLYDLGEFNQKGTVRTKYGTKAQYLQAIQAAHAAGMQVYADVVFDHKGGADGTEWVDAVEVNPSDRNQEISGTYQIQAWTKFDFPGRGNTYSSFKWRWYHFDGVDWDESRKLSRIYKFRGKAWDWEVDTEFGNYDYLMYADLDMDHPEVVTELKNWGKWYVNTTNIDGFRLDAVKHIKFSFFPDWLSYVRSQTGKPLFTVGEYWSYDINKLHNYITKTDGTMSLFDAPLHNKFYTASKSGGAFDMRTLMTNTLMKDQPTLAVTFVDNHDTEPGQALQSWVDPWFKPLAYAFILTRQEGYPCVFYGDYYGIPQYNIPSLKSKIDPLLIARRDYAYGTQHDYLDHSDIIGWTREGGTEKPGSGLAALITDGPGGSKWMYVGKQHAGKVFYDLTGNRSDTVTINSDGWGEFKVNGGSVSVWVPRKTTVSTIARPITTRPWTGEFVRWTEPRLVAWP

SEQ ID NO: 17 - protein, 481 aa, Bacillus licheniformis
  VARIANT (1)..(481)
VNGTLMQYFEWYTPNDGQHWKRLQNDAEHLSDIGITAVWIPPAYKGTSQADVGYGAYDLYDLGEFHQKGTVRTKYGTKGELQSAIKSLHSRDINVYGDVVINHKGGADATEDVTAVEVDPADRNRVISGEHLIKAWTHFHFPGRGSTYSDFKWYWYHFDGTDWDESRKLNRIYKFQGKTWDWEVSNEFGNYDYLMYADIDYDHPDVVAEIKRWGTWYANELQLDGFRLDAVKHIKFSFLRDWVNHVREKTGKEMFTVAEYWSNDLGALENYLNKTNFNHSVFDVPLHYQFHAASTQGGGYDMRKLLNGTVVSKHPLKSVTFVDNHDTQPGQSLESTVQTWFKPLAYAFILTRESGYPQVFYGDMYGTKGDSQREIPALKHKIEPILKARKQYAYGAQHDYFDHHDIVGWTREGDSSVANSGLAALITDGPGGAKRMYVGRQNAGETWHDITGNRSEPVVINSEGWGEFHVNGGSVSIYVQR

SEQ ID NO: 18 - protein, 483 aa, Bacillus sp.
  VARIANT (1)..(483)
HHNGTNGTMMQYFEWYLPNDGNHWNRLRSDASNLKDKGISAVWIPPAWKGASQNDVGYGAYDLYDLGEFNQKGTIRTKYGTRNQLQAAVNALKSNGIQVYGDVVMNHKGGADATEMVKAVEVNPNNRNQEVSGEYTIEAWTKFDFPGRGNTHSNFKWRWYHFDGVDWDQSRKLNNRIYKFRGKGWDWEVDTEFGNYDYLMYADIDMDHPEVVNELRNWGVWYTNTLGLDGFRIDAVKHIKYSFTRDWINHVRSATGKNMFAVAEFWKNDLGAIENYLNKTNWNHSVFDVPLHYNLYNASKSGGNYDMRQIFNGTVVQKHPMHAVTFVDNHDSQPEEALESFVEEWFKPLAYALTLTREQGYPSVFYGDYYGIPTHGVPAMKSKIDPILEARQKYAYGRQNDYLDHHNIIGWTREGNTAHPNSGLATIMSDGAGGNKWMFVGRNKAGQVWTDITGNKAGTVTINADGWGNFSVNGGSVSIWVNK

(19) United States
(12) Patent Application Publication
(10) Pub. No.: US 2008/0317726 A1
(43) Pub. Date: Dec. 25, 2008

(54) Proteases for Pharmaceutical Use

(75) Inventors: Allan Svendsen, Horsholm (DK); Claus Crone Fuglsang, Vekso (DK); Peter Colin Gregory, Hannover (DE)

Correspondence Address: NOVOZYMES NORTH AMERICA, INC., 500 FIFTH AVENUE, SUITE 1600, NEW YORK, NY, 10110, US

(73) Assignees: Novozymes A/S, Bagsvaerd (DK); Solvay Pharmaceuticals GmbH, Hannover (DE)

(21) Appl. No.: 11/917,595

(22) PCT Filed: Jun. 16, 2006
(86) PCT No.: PCT/DK2006/000353

§ 371 Date: Jan. 23, 2008

Related U.S. Application Data
(60) Provisional application No. 60/694,168, filed on Jun. 27, 2005.
(60) Provisional application No. 60/739,282, filed on Nov. 23, 2005.

(30) Foreign Application Priority Data
Jun. 24, 2005 (DK) PA 2005 00930
Nov. 23, 2005 (DK) PA 2005 01643

(51) Int. Cl. C12N 9/50; A61K 38/48; A61P 3/10; A61P 1/18; A61P 11/00; A61K 38/54

(52) U.S. Cl. 424/94.2; 435/219; 424/94.63

(57) ABSTRACT

The pharmaceutical use of proteases related to amino acids 1-274 of SEQ ID NO: 2, the serine protease derived from Bacillus licheniformis, which is also designated subtilisin Carlsberg, optionally in combination with a lipase and/or an amylase. Examples of medical indications are: Treatment of digestive disorders, pancreatic exocrine insufficiency (PEI), pancreatitis, cystic fibrosis, diabetes type I, and/or diabetes type II.

### TECHNICAL FIELD

The present invention relates to the pharmaceutical use of proteases related to a serine protease derived from Bacillus licheniformis (amino acids 1-274 of SEQ ID NO: 2), optionally in combination with a lipase and/or an amylase. Examples of medical indications are: Treatment of digestive disorders, pancreatic exocrine insufficiency (PEI), pancreatitis, cystic fibrosis, diabetes type I, and/or diabetes type II.

### BACKGROUND ART

Several commercial medicaments in the form of pancreatic enzyme supplements are known for the treatment of pancreatic exocrine insufficiency. The active ingredients of these products are digestive enzymes, mainly amylase, lipase and protease, which are normally produced in the pancreas and excreted to the upper part of the small intestine (the duodenum). The enzymes used in such medicaments derive from bovine or swine pancreas, however there are also products on the market with microbial enzymes, e.g. the product Nortase® which contains a lipase from Rhizopus oryzae, a protease from Aspergillus oryzae, and an amylase from Aspergillus oryzae.

U.S. Pat. No. 5,614,189 (EP 600868) describes the use of, i.a., a lipase derived from Humicola lanuginosa in pancreatic enzyme replacement therapy, for example in the treatment of patients suffering from cystic fibrosis. This lipase is from Humicola lanuginosa DSM 4109 and has the amino acid sequence of amino acids 1-269 of SEQ ID NO: 14.

WO 00/54799 describes the use of physiologically acceptable enzyme mixtures having lipolytic, proteolytic and amylolytic activity in the treatment of diabetes mellitus type I and II.

WO 02/060474 describes the use of a concentrated lipase from Rhizopus delemar, a neutral protease from Aspergillus melleus, and an amylase from Aspergillus oryzae in the treatment of maldigestion.

WO 01/62280 describes the use of certain a non-fungal lipase crystal crosslinked with a multifunctional crosslinking agent, a protease, and an amylase, wherein the lipase crystal is active at a pH range from about 2.0 to 9.0, for treating or preventing a gastrointestinal disorder in a mammal. A preferred lipase is from Pseudomonas, preferred amylases are from Bacillus or Aspergillus, preferred proteases are bromelain, papain or ficin.

EP 0828509 describes the use of certain acid-stable amylases, optionally in combination with certain acid-stable lipases and/or proteases, in the treatment of exocrine pancreas insufficiency. A preferred amylase is from Aspergillus niger, and preferred lipases are from Rhizopus arrhizus or Rhizopus javanicus.

WO 91/00345 describes a number of serine subtilisin proteases and improved variants thereof, for use in detergent compositions.

WO 2005/115445 (published after the priority dates of the present application) describes the pharmaceutical use of proteases related to a protease derived from Nocardiopsis sp. NRRL 18262 (this protease having the amino acid sequence of amino acids 1-188 of SEQ ID NO: 1 in this reference), optionally in combination with a lipase and/or an amylase. The medical indications are the same as in the present invention.

WO 02/077187 discloses variants of a Bacillus amyloliquefaciens subtilisin having an altered T-cell epitope and various uses thereof. Pharmaceutical compositions are claimed.

WO 01/12795 discloses the pharmaceutical use of proteolytic enzyme compositions. Preferred proteases are from Aspergillus oryzae, Aspergillus niger, Aspergillus sojae, Aspergillus flavus, Aspergillus awamori, or Bacillus subtilis.

WO 2004/078773 discloses how to maintain proteases such as subtilisin proteases in an inactive state which can be activated upon demand through an external signal. Among other uses the use of pro-subtilisin in wound cleaning formulations is disclosed, and how to cause active subtilisin to be formed. A preferred protease enzyme is Prop-subtilisin or Prop-loaded subtilisin (Yabuta et al., J. Biol. Chem. 278:15246-51, 2003).

US 2002/0081703 discloses a method for reducing allergenicity of non-human proteins, wherein an epitope is identified and replaced with an analogous region within a human subtilisin. Pharmaceutical compositions comprising a human subtilisin are claimed.

There is a need in the art for alternative, preferably improved, enzymes for pharmaceutical use, in particular for the medical indications mentioned above.

### SUMMARY OF THE INVENTION

The present invention provides alternative, preferably improved, enzymes for pharmaceutical use, in particular for the treatment of digestive disorders, pancreatic exocrine insufficiency (PEI), pancreatitis, cystic fibrosis, diabetes type I, and/or diabetes type II. The new enzymes are proteases, amylases, and lipases. Preferably, the enzymes for use according to the invention have an improved efficacy in vivo and/or in vitro; an improved pH-stability profile; an improved pH-activity profile; are stable against degradation by proteases; are stable in the presence of bile salts; and/or have a reduced allergenicity

The present invention relates to a protease of at least 50% identity to amino acids 1-274 of SEQ ID NO: 2, for use as a medicament, optionally in combination with a lipase, and/or an amylase.

The invention also relates to the use of such proteases for the manufacture of a medicament for the treatment of digestive disorders, PEI, pancreatitis (acute and/or chronic), cystic fibrosis, diabetes type I, and/or diabetes type II, these uses optionally further comprising the use of a lipase, and/or an amylase.

The invention furthermore relates to a pharmaceutical composition comprising such proteases, together with at least one pharmaceutically acceptable auxiliary material, optionally including a lipase and/or an amylase.

The invention also relates to a method for the treatment of digestive disorders, PEI, pancreatitis (acute and/or chronic), cystic fibrosis, diabetes type I, and/or diabetes type II, by administering a therapeutically effective amount of such proteases, optionally together with a lipase and/or an amylase.

### DETAILED DESCRIPTION OF THE INVENTION

### Enzymes

The present invention relates to the pharmaceutical use of proteases having at least 50% identity to the protease of amino acids 1-274 of SEQ ID NO: 2, a serine protease derived from Bacillus licheniformis, which is also designated subtilisin Carlsberg. The invention also relates to the use of such proteases for the manufacture of a medicament for the treatment of digestive disorders, PEI, pancreatitis, cystic fibrosis, diabetes type I, and/or diabetes type II. The invention furthermore relates to a pharmaceutical composition comprising such proteases, together with at least one pharmaceutically acceptable auxiliary material, as well as to a method for the treatment of the above-mentioned diseases, by administering a therapeutically effective amount of such proteases.

In what follows, the protease for use in the compositions, methods and uses of the invention is referred to as the “protease of the invention.”

In preferred embodiments, the protease of the invention has a degree of identity to amino acids 1-274 of SEQ ID NO: 2 of at least 51%, 52%, 53%, 54%, 55%, 56%, 57%, 58%, 59%, or at least 60%. In other preferred embodiments, the protease of the invention has a degree of identity to amino acids 1-274 of SEQ ID NO: 2 of at least 61%, 62%, 63%, 64%, 65%, 66%, 67%, 68%, 69%, or at least 70%. In still further preferred embodiments, the protease of the invention has a degree of identity to amino acids 1-274 of SEQ ID NO: 2 of at least 71%, 72%, 73%, 74%, 75%, 76%, 77%, 78%, 79%, or at least 80%. In additional preferred embodiments, the protease of the invention has a degree of identity to amino acids 1-274 of SEQ ID NO: 2 of at least 81%, 82%, 83%, 84%, 85%, 86%, 87%, 88%, 89%, or at least 90%. In most preferred embodiments, the protease of the invention has a degree of identity to amino acids 1-274 of SEQ ID NO: 2 of at least 91%, 92%, 93%, 94%, 95%, 96%, 97%, 98%, or at least 99%.

The term “protease” is defined herein as an enzyme that hydrolyses peptide bonds. It includes any enzyme belonging to the EC 3.4 enzyme group (including each of the thirteen subclasses thereof, these enzymes being in the following referred to as “belonging to the EC 3.4.-.-group”). The EC number refers to Enzyme Nomenclature 1992 from NC-IUBMB, Academic Press, San Diego, Calif., including supplements 1-5 published in Eur. J. Biochem. 1994, 223, 1-5; Eur. J. Biochem. 1995, 232, 1-6; Eur. J. Biochem. 1996, 237, 1-5; Eur. J. Biochem. 1997, 250, 1-6; and Eur. J. Biochem. 1999, 264, 610-650; respectively. The nomenclature is regularly supplemented and updated; see e.g. the World Wide Web at http://www.chem.qmw.ac.uk/iubmb/enzyme/index.html.

Proteases are classified on the basis of their catalytic mechanism into the following groups: Serine proteases (S), cysteine proteases (C), aspartic proteases (A), metallo proteases (M), and unknown, or as yet unclassified, proteases (U), see Handbook of Proteolytic Enzymes, A. J. Barrett, N. D. Rawlings, J. F. Woessner (eds), Academic Press (1998), (in what follows referred to as “the handbook”), in particular the general introduction part.

In another embodiment, the protease of the invention is a serine protease. The term serine protease refers to serine peptidases and their clans as defined in the handbook, see in particular chapters 1-175. A serine protease is a peptidase in which the catalytic mechanism depends upon the hydroxyl group of a serine residue acting as the nucleophile that attacks the peptide bond.

In a still further embodiment, the protease of the invention is a subtilisin and/or derived from the subtilisin family. The terms subtilisin or subtilisin family include all Clan SB serine proteases, in particular Family S8 thereof (Clan SB is dealt with in Chapter 93 of the handbook). For determining whether a given protease is a subtilisin or not, reference is made to the handbook and the principles indicated therein. Such determination can be carried out for all types of proteases, be it naturally occurring or wild-type proteases; or genetically engineered or synthetic proteases. In a particular embodiment, the order of the catalytic triad in the protease of the invention is Asp-His-Ser. In another particular embodiment, the tertiary structure of the protease of the invention includes both alpha-helices and beta sheets. Clan SB includes endopeptidases and exopeptidases. In a still further particular embodiment the protease of the invention is an endopeptidase. Endopeptidases show activity on N- and C-terminally blocked peptide substrates that are relevant for the specificity of the protease in question.

In a particular embodiment, the protease of the invention is not Prop-subtilisin or Prop-loaded subtilisin (Yabuta et al., J. Biol. Chem. 278:15246-51, 2003). In another particular embodiment the protease of the invention is not a wildtype Bacillus subtilis subtilisin, and/or not derived from Bacillus subtilis.

Accordingly, in a first aspect, the protease of the invention is selected from the group consisting of: (a) proteases belonging to the EC 3.4.-.-enzyme group; (b) serine proteases; (c) subtilisin proteases of peptidase Clan SB; and (d) subtilisin proteases of Family S8.

In a second aspect, the protease of the invention is derived from a microorganism, for example from a fungus, or from a bacterium. Examples of bacteria are strains of Bacillus, such as strains of Bacillus alkalophilus, Bacillus amyloliquefaciens, Bacillus brevis, Bacillus clausii, Bacillus circulans, Bacillus coagulans, Bacillus lautus, Bacillus lentus, Bacillus licheniformis, Bacillus megaterium, Bacillus mesentericus, Bacillus natto, Bacillus pumilus, Bacillus sp., Bacillus stearothermophilus, Bacillus subtilis, Bacillus subtilis var natto, or Bacillus thuringiensis; in particular strains of Bacillus amyloliquefaciens, Bacillus clausii, Bacillus lentus, Bacillus licheniformis, Bacillus mesentericus, Bacillus natto, Bacillus pumilus, Bacillus sp., Bacillus stearothermophilus, Bacillus subtilis, or Bacillus subtilis var natto; preferably a strain of Bacillus licheniformis. In this context, the term “derived from” includes enzymes obtainable, or obtained, from wildtype strains; as well as, preferably, variants thereof having at least one substitution, insertion, and/or deletion of at least one amino acid residue. The term variant also includes shufflants, hybrids, chimeric enzymes and consensus enzymes. The variants may have been produced by any method known in the art, such as site-directed mutagenesis, random mutagenesis, consensus derivation processes (EP 897985), and gene shuffling (WO 95/22625, WO 96/00343), etc.

The following are examples of proteases of the invention derived from strains of Bacillus and related to the protease of amino acids 1-274 of SEQ ID NO: 2: Swissprot subt_bacli accession no. P00780 (derived from Bacillus licheniformis, amino acids 1-274 of SEQ ID NO: 5); Swissprot subn_bacna accession no. P35835 (derived from Bacillus natto, amino acids 1-275 of SEQ ID NO: 6); Swissprot subt_bacpu accession no. P07518 (derived from Bacillus pumilus, amino acids 1-275 of SEQ ID NO: 7); Swissprot subt_bacsu accession no. P04189 (derived from Bacillus subtilis, amino acids 1-275 of SEQ ID NO: 8); Swissprot subt_bacst accession no. P29142 (derived from Bacillus stearothermophilus, amino acids 1-275 of SEQ ID NO: 9); Swissprot subt_bacam accession no. P00782 (derived from Bacillus amyloliquefaciens, amino acids 1-275 of SEQ ID NO: 10); Swissprot subs_bacle accession no. P29600 (derived from Bacillus lentus, amino acids 1-269 of SEQ ID NO: 11); Swissprot elya_baccs accession no. P41362 (derived from Bacillus clausii, amino acids 1-269 of SEQ ID NO: 12); and Swissprot elya_bacya accession no. P20724 (derived from Bacillus sp., amino acids 1-268 of SEQ ID NO: 13); as well as variants thereof, as defined above.

Additional particular examples of proteases of the invention are the proteases contained in the following commercial products: Purafect MA, Purafect, Purafect Ox (variant M222S), Purafect Prime (Y217L), Properase (S87N+S101G+V104N), FN3 (N76D+S103A+V1041), FN4 (S101G+S103A+V1041+G159D+A232V+Q236H+Q245R+N248D+N252K)—all preferably variants of the mature part of SEQ ID NO: 10 and commercially available from Genencor/Danisco; Blap (the mature part of SEQ ID NO: 11 with S99D+S101R+S103A+V1041+G160S), BLAP R (Blap with S3T+V41+V199M+V2051+L217D), and BLAP X (Blap with S3T+V41+V205I)—all from Henkel/Kemira; and KAP (A230V+S256G+S259N) from Kao.

In a third aspect, the protease of the invention is, or can be seen as, a variant of the protease of SEQ ID NO: 2, i.e. it comprises at least one substitution, deletion, and/or insertion of one or more amino acids of amino acids 1-274 of SEQ ID NO: 2. Preferably, the amino acid changes are of a minor nature, that is conservative amino acid substitutions or insertions that do not significantly affect the folding and/or activity of the protein; small deletions; small amino- or carboxyl-terminal extensions, such as an amino-terminal methionine residue; a small linker peptide; or a small extension that facilitates purification by changing net charge or another function, such as a poly-histidine tract, an antigenic epitope or a binding domain. In this context, the term “small” independently designates a number of up to 25 amino acid residues. In preferred embodiments, the term “small” independently designates up to 24, 23, 22, 21, or up to 20 amino acid residues. In additional preferred embodiments, the term “small” independently designates up to 19, 18, 17, 16, 15, 14, 13, 12, 11, or up to 10 amino acid residues. In further preferred embodiments, the term “small” independently designates up to 9, 8, 7, 6, 5, 4, 3, 2, or up to 1 amino acid residue. In alternative embodiments, the term “small” independently designates up to 40, 39, 38, 37, 36, 35, 34, 33, 32, 31, 30, 29, 28, 27, or up to 26 amino acid residues.

Examples of conservative substitutions are within the group of basic amino acids (arginine, lysine and histidine), acidic amino acids (glutamic acid and aspartic acid), polar amino acids (serine, threonine, glutamine and asparagine), hydrophobic amino acids (leucine, isoleucine, valine and alanine), aromatic amino acids (phenylalanine, tryptophan and tyrosine), and small amino acids (glycine, alanine, proline, serine, threonine, cysteine and methionine).

In the alternative, examples of conservative substitutions are within the group of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions which do not generally alter specific activity are known in the art and are described, for example, by H. Neurath and R. L. Hill, 1979, In, The Proteins, Academic Press, New York. The most commonly occurring exchanges are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Preferred variants of any of the mature protease parts of SEQ ID NOs: 5-13, such as, e.g., amino acids 1-268 of SEQ ID NO: 13, comprise at least one substitution, deletion, and/or insertion of one or more amino acids (as compared to the parent, or ancestor, enzyme such as, e.g., amino acids 1-268 of SEQ ID NO: 13), as explained above for variants of amino acids 1-274 of SEQ ID NO: 2. More preferably these variants are with conservative amino acid substitutions or insertions, small deletions, small linkers, or with small extensions, as also explained in detail above for variants of amino acids 1-274 of SEQ ID NO: 2. A specific example of a protease variant of the invention is variant 99aE of SEQ ID NO: 11 (see Example 4).

In a fourth aspect, the protease of the invention has an amino acid sequence which differs by no more than 25, 24, 23, 22, 21, 20, 19, 18, 17, 16, 15, 14, 13, 12, or no more than 11 amino acids from amino acids 1-274 of SEQ ID NO: 2; or, it differs from amino acids 1-274 of SEQ ID NO: 2 by no more than 10, 9, 8, 7, 6, 5, 4, 3, 2, or by no more than 1 amino acid. In alternative embodiments, the protease of the invention has an amino acid sequence which differs by no more than 40, 39, 38, 37, 36, 35, 34, 33, 32, 31, 30, 29, 28, 27, or no more than 26 amino acids from amino acids 1-274 of SEQ ID NO: 2.

Preferred variants of any of the mature protease parts of SEQ ID NOs: 5-13, such as, e.g. amino acids 1-275 of SEQ ID NO: 7, have an amino acid sequence which differ by no more than 25, 24, 23, 22, 21, 20, 19, 18, 17, 16, 15, 14, 13, 12, or no more than 11 amino acids from the mature parts of any one of SEQ ID NOs: 5-13, such as, e.g., amino acids 1-275 of SEQ ID NO: 7; or, they differ from the mature parts of any one of SEQ ID NOs: 5-13, such as, e.g., amino acids 1-275 of SEQ ID NO: 7, by no more than 10, 9, 8, 7, 6, 5, 4, 3, 2, or by no more than 1 amino acid.

In a fifth aspect, the protease of the invention is an allelic variant of SEQ ID NO: 2 (preferably an allelic variant of the mature part thereof), an allelic variant of any one of SEQ ID NOs. 5-13 (preferably an allelic variant of any one of the mature parts thereof), or a fragment of any of these that has protease activity. The term allelic variant denotes any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene. The term fragment is defined herein as a polypeptide having one or more amino acids deleted from the amino and/or carboxyl terminus of amino acids 1-274 of SEQ ID NO: 2, or, from the amino and/or carboxyl terminus of any one of SEQ ID NOs: 5-13, preferably from the mature parts thereof. Preferably, a small number of amino acids has been deleted, small being defined as explained above. More preferably, a fragment contains at least 244, 245, 246, 247, 248, 249, or at least 250 amino acid residues. Most preferably, a fragment contains at least 251, 252, 253, 254, 255, 256, 257, 258, 259, 260, 261, 262, 263, 264, 265, 266, 267, 268, 269, 270, 271, 272, or at least 273 amino acid residues.

In summary, one embodiment of the present invention relates to a protease for pharmaceutical use, wherein a) the protease comprises an amino acid sequence selected from the group consisting of amino acids 1-274 of SEQ ID NO: 2, amino acids 1-274 of SEQ ID NO: 5, amino acids 1-275 of SEQ ID NO: 6, amino acids 1-275 of SEQ ID NO: 7, amino acids 1-275 of SEQ ID NO: 8, amino acids 1-275 of SEQ ID NO: 9, amino acids 1-275 of SEQ ID NO: 10, amino acids 1-269 of SEQ ID NO: 11, amino acids 1-269 of SEQ ID NO: 12, and amino acids 1-268 of SEQ ID NO: 13; and/or b) the protease is a variant of an amino acid sequence selected from the group consisting of amino acids 1-274 of SEQ ID NO: 2, amino acids 1-274 of SEQ ID NO: 5, amino acids 1-275 of SEQ ID NO: 6, amino acids 1-275 of SEQ ID NO: 7, amino acids 1-275 of SEQ ID NO: 8, amino acids 1-275 of SEQ ID NO: 9, amino acids 1-275 of SEQ ID NO: 10, amino acids 1-269 of SEQ ID NO: 11, amino acids 1-269 of SEQ ID NO: 12, and amino acids 1-268 of SEQ ID NO: 13, wherein the variant differs from the respective amino acid sequence by no more than twenty-five amino acids, and wherein: (i) the variant comprises at least one substitution, deletion and/or insertion of one or more amino acids as compared to the respective amino acid sequence; and/or (ii) the variant comprises at least one small deletion as compared to the respective amino acid sequence; and/or (iii) the variant comprises at least one small N- or C-terminal extension as compared to the respective amino acid sequence; and/or c) the protease is an allelic variant of a protease having amino acids selected from the group consisting of amino acids 1-274 of SEQ ID NO: 2, amino acids 1-274 of SEQ ID NO: 5, amino acids 1-275 of SEQ ID NO: 6, amino acids 1-275 of SEQ ID NO: 7, amino acids 1-275 of SEQ ID NO: 8, amino acids 1-275 of SEQ ID NO: 9, amino acids 1-275 of SEQ ID NO: 10, amino acids 1-269 of SEQ ID NO: 11, amino acids 1-269 of SEQ ID NO: 12, and amino acids 1-268 of SEQ ID NO: 13; and/or d) the protease is a fragment of a protease having amino acids selected from the group consisting of amino acids 1-274 of SEQ ID NO: 2, amino acids 1-274 of SEQ ID NO: 5, amino acids 1-275 of SEQ ID NO: 6, amino acids 1-275 of SEQ ID NO: 7, amino acids 1-275 of SEQ ID NO: 8, amino acids 1-275 of SEQ ID NO: 9, amino acids 1-275 of SEQ ID NO: 10, amino acids 1-269 of SEQ ID NO: 11, amino acids 1-269 of SEQ ID NO: 12, and amino acids 1-268 of SEQ ID NO: 13.

In particular, the present inventions relates to a protease for pharmaceutical use, wherein the protease has an amino acid sequence selected from the group consisting of amino acids 1-274 of SEQ ID NO: 2, amino acids 1-274 of SEQ ID NO: 5, amino acids 1-275 of SEQ ID NO: 6, amino acids 1-275 of SEQ ID NO: 7, amino acids 1-275 of SEQ ID NO: 8, amino acids 1-275 of SEQ ID NO: 9, amino acids 1-275 of SEQ ID NO: 10, amino acids 1-269 of SEQ ID NO: 11, amino acids 1-269 of SEQ ID NO: 12, and amino acids 1-268 of SEQ ID NO: 13.

In another particular embodiment, the protease of the invention may be used in combination with an additional protease. Examples of additional proteases are mammalian proteases, and microbial proteases. A preferred mammalian protease is pancreas extract, e.g. from swine or ox, such as pancreatin. The pancreatin may be used in the form of an uncoated (raw) product, or in the form of a formulated product (enteric coated (to provide resistance against gastric acid), or non-functionally coated (coated, but not to provide resistance against gastric acid)). Pancreatin potentially comprises still further enzymatic active constituents like pancreatic lipase, BSSL (Bile Salt Stimulated Lipase), and/or pancreatic amylase. Preferred microbial proteases derive from bacterial or fungal strains, for example from a strain of Aspergillus, such as Aspergillus oryzae or Aspergillus melleus, in particular the product Prozyme 6™ (neutral, alkaline protease EC 3.4.21.63) which is commercially available from Amano Pharmaceuticals, Japan.

Optionally, the protease of the invention is used in combination with a lipase, with or without and amylase, as explained further below.

In the present context, a lipase means a carboxylic ester hydrolase EC 3.1.1.-, which includes activities such as EC 3.1.1.3 triacylglycerol lipase, EC 3.1.1.4 phospholipase A1, EC 3.1.1.5 lysophospholipase, EC 3.1.1.26 galactolipase, EC 3.1.1.32 phospholipase A1, EC 3.1.1.73 feruloyl esterase. In a particular embodiment, the lipase is an EC 3.1.1.3 triacyl-glycerol lipase.

In particular embodiments, the lipase is a mammalian lipase, e.g. pancreas extract from swine or ox, such as pancreatin. The pancreatin may be used in the form of an uncoated (raw) product, or in the form of a formulated product (enteric coated, or non-functionally coated, as defined above). Pancreatin potentially comprises still further enzymatic active constituents like pancreatic protease, BSSL (Bile Salt Stimulated Lipase), and/or pancreatic amylase. The lipase may also be a microbial lipase, for example derived from bacterial or fungal strains, such as Bacillus, Pseudomonas, Aspergillus, or Rhizopus. The lipase may in particular be derived from a strain of Rhizopus, such as Rhizopus javanicus, Rhizopus oryzae, or Rhizopus delemar, for example the product Lipase D Amano 2000™ (also designated Lipase D2™) which is commercially available from Amano Pharmaceuticals, Japan.

In further particular embodiments, the lipase is a recombinantly produced microbial lipase, for example derived from a fungus such as Humicola or Rhizomucor, from a yeast such as Candida, or from a bacterium such as Pseudomonas. In a preferred embodiment, the lipase is derived from a strain of Humicola lanuginosa or Rhizomucor miehei.

The Humicola lanuginosa (synonym Thermomyces lanuginosus) lipase (SEQ ID NO: 14) is described in EP 305216, and particular lipase variants are described in, for example, WO 92/05249, WO 92/19726, WO 94/25577, WO 95/22615, WO 97/04079, WO 97/07202, WO 99/42566, WO 00/32758, WO 00/60063, WO 01/83770, WO 02/055679, and WO 02/066622. A preferred Humicola lanuginosa lipase variant is a lipase comprising amino acids 1-269, or 2-269, of SEQ ID NO: 15, such as the following: (i) amino acids +1 to +269 of SEQ ID NO: 15, (ii) amino acids −5 to +269 of SEQ ID NO: 15, (iii) amino acids −4 to +269 of SEQ ID NO: 15; (iv) amino acids −3 to +269 of SEQ ID NO: 15; (v) amino acids −2 to +269 of SEQ ID NO: 15; (vi) amino acids −1 to +269 of SEQ ID NO: 15, (vii) amino acids +2 to +269 of SEQ ID NO: 15, as well as (viii) any mixture of two or more of the lipases of (i)-(vii)—as well as variants thereof. In a particular embodiment, the lipase is selected from the lipases of (i), (ii), and any mixture of (i) and (ii). Preferred mixtures of (i) and (ii) comprise at least 5%, preferably at least 10%, 20%, 30%, 40%, 50%, 60%, 70%, 80%, 90%, or at least 95% of lipase (i), the percentages being determined by N-terminal sequencing using the Edman method, as described in Example 5 of the PCT application claiming priority from DK patent application 2005 00929). Other preferred mixtures are: (a) compositions comprising 35-75%, preferably 40-70%, more preferably 45-65% of lipase (ii); (b) compositions comprising 20-60%, preferably 25-55%, more preferably 30-50%, most preferably 35-47% of lipase (i); (c) compositions comprising up to 30%, preferably up to 25%, more preferably up to 20%, most preferably up to 16% of lipase (vii); and (d) any combination of (a), (b), and/or (c), such as a composition comprising 45-65% of lipase (ii), 35-47% of lipase (i), and up to 16% of lipase (vii).

The lipases of SEQ ID NO: 14 and 15 may, e.g., be prepared as described in U.S. Pat. No. 5,869,438 (SEQ ID NO: 1 in the US patent referred to is a DNA sequence encoding the lipase of SEQ ID NO: 14). The lipase of SEQ ID NO: 15 may, e.g., be prepared by recombinant expression in a suitable host cell of a DNA sequence which is a modification of SEQ ID NO:1 of the US patent, the modification reflecting the amino acid differences between SEQ ID NO: 14 and 15 herein. Such modifications can be made by site-directed mutagenesis, as is known in the art.

Still further examples of fungal lipases are the cutinase from Humicola insolens which is described in EP 785994, and the phospholipase from Fusarium oxysporum which is described in EP 869167. Examples of yeast lipases are lipase A and B from Candida antarctica of which lipase A is described in EP 652945, and lipase B is described by, for example, Uppenberg et al in Structure, 2 (1994), 293. An example of a bacterial lipase is the lipase derived from Pseudomonas cepacia, which is described in EP 214761.

In a preferred embodiment, the lipase is at least 70% identical to the lipase of SEQ ID NO: 15, preferably amino acids 1-269 thereof. In additional preferred embodiments, the degree of identity to SEQ ID NO: 15, preferably amino acids 1-269 thereof, is at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99%. In alternative embodiments, the degree of identity to SEQ ID NO: 15, preferably amino acids 1-269 thereof, is at least about 50%, 51%, 52%, 53%, 54%, 55%, 56%, 57%, 58%, 59%, 60%, 61%, 62%, 63%, 64%, 65%, 66%, 67%, 68%, or at least 69%.

In a still further preferred embodiment, the lipase, like the mammalian pancreatic lipase, is a 1,3-position specific lipase.

Optionally, the protease of the invention, with or without a lipase as described above, is used in combination with an amylase.

In the present context, an amylase is an enzyme that catalyzes the endo-hydrolysis of starch and other linear and branched oligo- and polysaccharides. The amylose part of starch is rich in 1,4-alpha-glucosidic linkages, while the amylopectin part is more branched containing not only 1,4-alpha- but also 1,6-alpha-glucosidic linkages. In a particular embodiment, the amylase is an enzyme belonging to the EC 3.2.1.1 group.

In particular embodiments, the amylase is a mammalian amylase, e.g. pancreas extract from swine or ox, such as pancreatin. The pancreatin may be used in the form of an uncoated (raw) product, or in the form of a formulated product (enteric coated, or non-functionally coated, as defined above). Pancreatin potentially comprises still further enzymatic active constituents like pancreatic protease, BSSL, and/or pancreatic lipase. The amylase may also be a microbial amylase, for example derived from bacterial or fungal strains, such as Bacillus, Pseudomonas, Aspergillus, or Rhizopus.

The amylase may in particular be derived from a strain of Aspergillus, such as Aspergillus niger, Aspergillus oryzae or Aspergillus melleus, for example either of the products Amylase A1™ derived from Aspergillus oryzae which is commercially available from Amano Pharmaceuticals, Japan, or Amylase EC™ derived from Aspergillus melleus which is commercially available from Extract-Chemie, Germany.

Other examples of fungal amylases are the Aspergillus niger amylase (SWISSPROT P56271), which is also described in Example 3 of WO 89/01969, and the Aspergillus oryzae amylase. Examples of variants of the Aspergillus oryzae amylase are described in WO 01/34784.

The alpha-amylase derived from Bacillus licheniformis is an example of a bacterial alpha-amylase. This amylase is, for example, described in WO 99/19467, together with other homologous bacterial alpha-amylases derived from, for example, Bacillus amyloliquefaciens, and Bacillus stearothermophilus, as well as variants thereof. Examples of additional amylase variants are those described in U.S. Pat. No. 4,933,279; EP 722490, and EP 904360.

Preferred amylases are an amylase comprising amino acids 1-481 of SEQ ID NO: 16 (such as amino acids 1-481, 1-484, or 1-486 thereof), amino acids 1-481 of SEQ ID NO: 17, and/or amino acids 1-483 of SEQ ID NO: 18. In a preferred embodiment, the amylase is at least 70% identical to either of (i) amino acids 1-481 of SEQ ID NO: 16, (ii) amino acids 1-481 of SEQ ID NO: 17, and/or (iii) amino acids 1-483 of SEQ ID NO: 18. The amylases of SEQ ID NOs: 16-18 may, e.g., be prepared as described in co-pending DK application no. 2005 00931 entitled “Amylases for Pharmaceutical Use” and filed on Jun. 24, 2005 by Solvay Pharmaceuticals GmbH and Novozymes A/S.

In additional preferred embodiments of either of (i), (ii), or (iii), the degrees of identity to the respective parts of SEQ ID NO: 16, 17 or 18 is at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99%. In alternative embodiments, the degree of identity to the respective parts of SEQ ID NO: 16, 17 or 18 is at least about 50%, 51%, 52%, 53%, 54%, 55%, 56%, 57%, 58%, 59%, 60%, 61%, 62%, 63%, 64%, 65%, 66%, 67%, 68%, or at least 69%.

For the purposes of the present invention, particularly preferred combinations of enzymes are the following: (i) The protease of amino acids 1-274 of SEQ ID NO: 2 in combination with a lipase comprising amino acids 1-269, or 2-269, of SEQ ID NO: 15; (ii) the protease of amino acids 1-274 of SEQ ID NO: 2 in combination with an amylase comprising amino acids 1-481 of SEQ ID NO: 16 (such as amino acids 1-481, 1-484, or 1-486 thereof); (iii) the protease of amino acids 1-274 of SEQ ID NO: 2 in combination with the amylase having amino acids 1-481 of SEQ ID NO: 17; (iv) the protease of amino acids 1-274 of SEQ ID NO: 2 in combination with the amylase having amino acids 1-483 of SEQ ID NO: 18; (v) the protease of amino acids 1-274 of SEQ ID NO: 2 in combination with an amylase comprising amino acids 1-481 of SEQ ID NO: 16 (such as amino acids 1-481, 1-484, or 1-486 thereof), and a lipase comprising amino acids 1-269, or 2-269, of SEQ ID NO: 15; (vi) the protease of amino acids 1-274 of SEQ ID NO: 2 in combination with the amylase having amino acids 1-481 of SEQ ID NO: 17 and a lipase comprising amino acids 1-269, or 2-269, of SEQ ID NO: 15; and (vii) the protease of amino acids 1-274 of SEQ ID NO: 2 in combination with the amylase having amino acids 1-483 of SEQ ID NO: 18 and a lipase comprising amino acids 1-269, or 2-269, of SEQ ID NO: 15.

Accordingly, one embodiment of the present invention relates to a protease in combination with a lipase and/or an amylase for pharmaceutical use, wherein (i) the protease is a protease as defined herein; (ii) the lipase comprises amino acids 2-269 of SEQ ID NO: 15; and (iii) the amylase is an amylase selected from the group consisting of: a) an amylase comprising amino acids 1-481 of SEQ ID NO: 16, b) an amylase having amino acids 1-481 of SEQ ID NO: 17, and c) an amylase having amino acids 1-483 of SEQ ID NO: 18.

In particular, the present invention relates to a protease in combination with a lipase and/or an amylase for pharmaceutical use, wherein (i) the protease comprises or preferably is, or has, amino acids 1-274 of SEQ ID NO: 2; (ii) the lipase comprises amino acids 2-269 of SEQ ID NO: 15; and (iii) the amylase is an amylase selected from the group consisting of: a) an amylase comprising amino acids 1-481 of SEQ ID NO: 16, b) an amylase having amino acids 1-481 of SEQ ID NO: 17, and c) an amylase having amino acids 1-483 of SEQ ID NO: 18.

Other preferred combinations of enzymes are the following: (i) A protease having at least 50% identity to amino acids 1-274 of SEQ ID NO: 2 in combination with a lipase having at least 50% identity to amino acids 1-269 of SEQ ID NO: 15; (ii) a protease having at least 50% identity to amino acids 1-274 of SEQ ID NO: 2 in combination with an amylase having at least 50% identity to amino acids 1-481 of SEQ ID NO: 16; (iii) a protease having at least 50% identity to amino acids 1-274 of SEQ ID NO: 2 in combination with an amylase having at least 50% identity to amino acids 1-481 of SEQ ID NO: 17; (iv) a protease having at least 50% identity to amino acids 1-274 of SEQ ID NO: 2 in combination with an amylase having at least 50% identity to amino acids 1-483 of SEQ ID NO: 18; (v) a protease having at least 50% identity to amino acids 1-274 of SEQ ID NO: 2 in combination with an amylase having at least 50% identity to amino acids 1-481 of SEQ ID NO: 16, and a lipase having at least 50% identity to amino acids 1-269 of SEQ ID NO: 15; (vi) a protease having 50% identity to amino acids 1-274 of SEQ ID NO: 2 in combination with an amylase having at least 50% identity to amino acids 1-481 of SEQ ID NO: 17 and a lipase having at least 50% identity to amino acids 1-269 of SEQ ID NO: 15; and (vii) a protease having at least 50% identity to amino acids 1-274 of SEQ ID NO: 2 in combination with an amylase having at least 50% identity to amino acids 1-483 of SEQ ID NO: 18 and a lipase having at least 50% identity to amino acids 1-269 of SEQ ID NO: 15. In preferred embodiments of (i)-(vii), each degree of identity is, independently, at least 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99%.

Accordingly, one embodiment of the present invention relates to a protease in combination with a lipase and/or an amylase for pharmaceutical use, wherein (i) the protease is selected from the group of a) a protease having at least 50% identity to amino acids 1-274 of SEQ ID NO: 2; b) a protease comprising an amino acid sequence selected from the group consisting of amino acids 1-274 of SEQ ID NO: 2, amino acids 1-274 of SEQ ID NO: 5, amino acids 1-275 of SEQ ID NO: 6, amino acids 1-275 of SEQ ID NO: 7, amino acids 1-275 of SEQ ID NO: 8, amino acids 1-275 of SEQ ID NO: 9, amino acids 1-275 of SEQ ID NO: 10, amino acids 1-269 of SEQ ID NO: 11, amino acids 1-269 of SEQ ID NO: 12, and amino acids 1-268 of SEQ ID NO: 13; c) a protease being a variant of an amino acid sequence selected from the group consisting of amino acids 1-274 of SEQ ID NO: 2, amino acids 1-274 of SEQ ID NO: 5, amino acids 1-275 of SEQ ID NO: 6, amino acids 1-275 of SEQ ID NO: 7, amino acids 1-275 of SEQ ID NO: 8, amino acids 1-275 of SEQ ID NO: 9, amino acids 1-275 of SEQ ID NO: 10, amino acids 1-269 of SEQ ID NO: 11, amino acids 1-269 of SEQ ID NO: 12, and amino acids 1-268 of SEQ ID NO: 13, wherein the variant differs from the respective amino acid sequence by no more than twenty-five amino acids, and wherein: (i) the variant comprises at least one substitution, deletion and/or insertion of one or more amino acids as compared to the respective amino acid sequence; and/or (ii) the variant comprises at least one small deletion as compared to the respective amino acid sequence; and/or (iii) the variant comprises at least one small N- or C-terminal extension as compared to the respective amino acid sequence; d) a protease being an allelic variant of a protease having amino acids selected from the group consisting of amino acids 1-274 of SEQ ID NO: 2, amino acids 1-274 of SEQ ID NO: 5, amino acids 1-275 of SEQ ID NO: 6, amino acids 1-275 of SEQ ID NO: 7, amino acids 1-275 of SEQ ID NO: 8, amino acids 1-275 of SEQ ID NO: 9, amino acids 1-275 of SEQ ID NO: 10, amino acids 1-269 of SEQ ID NO: 11, amino acids 1-269 of SEQ ID NO: 12, and amino acids 1-268 of SEQ ID NO: 13; e) a protease being a fragment of a protease having amino acids selected from the group consisting of amino acids 1-274 of SEQ ID NO: 2, amino acids 1-274 of SEQ ID NO: 5, amino acids 1-275 of SEQ ID NO: 6, amino acids 1-275 of SEQ ID NO: 7, amino acids 1-275 of SEQ ID NO: 8, amino acids 1-275 of SEQ ID NO: 9, amino acids 1-275 of SEQ ID NO: 10, amino acids 1-269 of SEQ ID NO: 11, amino acids 1-269 of SEQ ID NO: 12, and amino acids 1-268 of SEQ ID NO: 13; and f) a protease having an amino acid sequence selected from the group consisting of amino acids 1-274 of SEQ ID NO: 2, amino acids 1-274 of SEQ ID NO: 5, amino acids 1-275 of SEQ ID NO: 6, amino acids 1-275 of SEQ ID NO: 7, amino acids 1-275 of SEQ ID NO: 8, amino acids 1-275 of SEQ ID NO: 9, amino acids 1-275 of SEQ ID NO: 10, amino acids 1-269 of SEQ ID NO: 11, amino acids 1-269 of SEQ ID NO: 12, and amino acids 1-268 of SEQ ID NO: 13; (ii) the lipase has at least 70% identity to a lipase having amino acids 1-269 of SEQ ID NO: 15; and (iii) the amylase has at least 70% identity to an amylase selected from the group consisting of: a) an amylase having amino acids 1-481 of SEQ ID NO: 16, b) an amylase having amino acids 1-481 of SEQ ID NO: 17, and c) an amylase having amino acids 1-483 of SEQ ID NO: 18.

Generally, the protease, lipase, and amylase enzymes (hereinafter “the enzyme(s),” viz. the enzymes of the invention) may be natural or wild-type enzymes (obtained from animals, in particular mammals, for example human or swine enzymes; from plants, or from microorganisms), but also any mutants, variants, fragments etc. thereof exhibiting the desired enzyme activity, as well as synthetic enzymes, such as shuffled, hybrid, or chimeric enzymes, and consensus enzymes.

In a specific embodiment, the enzyme(s) are low-allergenic variants, designed to invoke a reduced immunological response when exposed to animals, including man. The term immunological response is to be understood as any reaction by the immune system of an animal exposed to the enzyme(s). One type of immunological response is an allergic response leading to increased levels of IgE in the exposed animal. Low-allergenic variants may be prepared using techniques known in the art. For example the enzyme(s) may be conjugated with polymer moieties shielding portions or epitopes of the enzyme(s) involved in an immunological response. Conjugation with polymers may involve in vitro chemical coupling of polymer to the enzyme(s), e.g. as described in WO 96/17929, WO 98/30682, WO 98/35026, and/or WO 99/00489. Conjugation may in addition or alternatively thereto involve in vivo coupling of polymers to the enzyme(s). Such conjugation may be achieved by genetic engineering of the nucleotide sequence encoding the enzyme(s), inserting consensus sequences encoding additional glycosylation sites in the enzyme(s) and expressing the enzyme(s) in a host capable of glycosylating the enzyme(s), see e.g. WO 00/26354. Another way of providing low-allergenic variants is genetic engineering of the nucleotide sequence encoding the enzyme(s) so as to cause the enzymes to self-oligomerize, effecting that enzyme monomers may shield the epitopes of other enzyme monomers and thereby lowering the antigenicity of the oligomers. Such products and their preparation is described e.g. in WO 96/16177. Epitopes involved in an immunological response may be identified by various methods such as the phage display method described in WO 00/26230 and WO 01/83559, or the random approach described in EP 561907. Once an epitope has been identified, its amino acid sequence may be altered to produce altered immunological properties of the enzyme(s) by known gene manipulation techniques such as site directed mutagenesis (see e.g. WO 00/26230, WO 00/26354 and/or WO 00/22103) and/or conjugation of a polymer may be done in sufficient proximity to the epitope for the polymer to shield the epitope.

In particular embodiments, the enzyme(s) are (i) stable at pH 2-8, preferably also at pH 3-7, more preferably at pH 4-6; (ii) active at pH 4-9, preferably 4-8; (iii) stable against degradation by pepsin and other digestive proteases (such as pancreas proteases, i.e., mainly trypsin and chymotrypsin); and/or (iv) stable and/or active in the presence of bile salts.

Preferably, the protease of the invention is acid-stable, which means that the pure protease enzyme remains active even after continued exposure to an acid environment. Preferably, the remaining activity is a factor 1.1, 1.2, 1.3, 1.5, 1.6, 1.8, 2.0, 2.5, and 3.0 higher than the remaining activity of a comparable protease already known for pharmaceutical purposes.

In further particular embodiments, the acid-stability means that the protease activity of the pure protease enzyme, in a dilution corresponding to A₂₈₀=1.0, and following incubation for 2 hours at 37° C. in the following buffer (100 mM succinic acid, 100 mM HEPES, 100 mM CHES, 100 mM CABS, 1 mM CaCl₂, 150 mM KCl, 0.01% Triton® X-100, pH 3.5) is at least 40% (or at least 45, 50, 55, 60, 65, 70, 75, 80, 85, 90, 95, or at least 97%) of the reference activity, as measured using the assay described in Example 2C of WO 01/58276 (substrate: Suc-AAPF-pNA, pH 9.0, 25° C.). The term reference activity refers to the protease activity of the same protease, following incubation in pure form, in a dilution corresponding to A₂₈₀=1.0, for 2 hours at 5° C. in the following buffer (100 mM succinic acid, 100 mM HEPES, 100 mM CHES, 100 mM CABS, 1 mM CaCl₂, 150 mM KCl, 0.01% Triton® X-100, pH 9.0) wherein the activity is determined as described above. The term A₂₈₀=1.0 means such concentration (dilution) of said pure protease which gives rise to an absorption of 1.0 at 280 nm in a 1 cm path length cuvette relative to a buffer blank. The term pure protease refers to a sample with a A₂₈₀/A₂₆₀ ratio above or equal to 1.70 (see Example 2E of WO 01/58276), and which by a scan of a Coomassie stained SDS-PAGE gel is measured to have at least 95% of its scan intensity in the band corresponding to said protease (see Example 2A of WO 01/58276).

The term “in combination with” refers to the combined use according to the invention of the protease, lipase and/or amylase. The combined use can be simultaneous, overlapping, or sequential, these three terms being generally interpreted in the light of the prescription made by the physician.

The term “simultaneous” refers to circumstances under which the enzymes are active at the same time, for example when they are administered at the same time as one or more separate pharmaceutical products, or if they are administered in one and the same pharmaceutical composition.

The term “sequential” refers to such instances where one and/or two of the enzymes are acting first, and the second and/or third enzyme subsequently. A sequential action can be obtained by administering the enzymes in question as separate pharmaceutical formulations with desired intervals, or as one pharmaceutical composition in which the enzymes in question are differently formulated (compartmentalized), for example with a view to obtaining a different release time, providing an improved product stability, or to optimizing the enzyme dosage.

The term “overlapping” refers to such instances where the enzyme activity periods are neither completely simultaneous nor completely sequential, viz. there is a certain period in which the enzymes are both, or all, active.

The term “a”, for example when used in the context of the enzyme(s) of the invention, means at least one. In particular embodiments, “a” means “one or more,” or “at least one”, which again means one, two, three, four, five etc.

The relatedness between two amino acid sequences is described by the parameter “identity”.

For purposes of the present invention, the alignment of two amino acid sequences is determined by using the Needle program from the EMBOSS package (http://emboss.org) version 2.8.0. The Needle program implements the global alignment algorithm described in Needleman, S. B. and Wunsch, C. D. (1970) J. Mol. Biol. 48, 443-453. The substitution matrix used is BLOSUM62, gap opening penalty is 10, and gap extension penalty is 0.5.

The degree of identity between an amino acid sequence of the present invention (“invention sequence”; e.g. amino acids 1-274 of SEQ ID NO: 2) and a different amino acid sequence (“foreign sequence”; e.g. amino acids 1-188 of SEQ ID NO: 1 of WO 2005/115445) is calculated as the number of exact matches in an alignment of the two sequences, divided by the length of the “invention sequence” or the length of the “foreign sequence”, whichever is the shortest. The result is expressed in percent identity.

An exact match occurs when the “invention sequence” and the “foreign sequence” have identical amino acid residues in the same positions of the overlap (in the alignment example below this is represented by “I”). The length of a sequence is the number of amino acid residues in the sequence (e.g. the length of SEQ ID NO: 2 is 274).

In the, purely hypothetical, alignment example below, the overlap is the amino acid sequence “HTWGER-NL” of Sequence 1; or the amino acid sequence “HGWGEDANL” of Sequence 2. In the example a gap is indicated by a “-”.

Hypothetical Alignment Example:

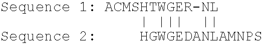

Accordingly, the percentage of identity of Sequence 1 to Sequence 2 is 6/12=0.5, corresponding to 50%.

In a particular embodiment, the percentage of identity of an amino acid sequence of a polypeptide with, or to, amino acids 1-274 of SEQ ID NO: 2 is determined by i) aligning the two amino acid sequences using the Needle program, with the BLOSUM62 substitution matrix, a gap opening penalty of 10, and a gap extension penalty of 0.5; ii) counting the number of exact matches in the alignment; iii) dividing the number of exact matches by the length of the shortest of the two amino acid sequences, and iv) converting the result of the division of iii) into percentage.

In the alternative, the degree of identity between two amino acid sequences may be determined by the program “align” which is a Needleman-Wunsch alignment (i.e. a global alignment). The sequences are aligned by the program, using the default scoring matrix BLOSUM50. The penalty for the first residue of a gap is 12, and for further residues of a gap the penalties are 2. The Needleman-Wunsch algorithm is described in Needleman, S. B. and Wunsch, C. D., (1970), Journal of Molecular Biology, 48: 443-453, and the align program by Myers and W. Miller in “Optimal Alignments in Linear Space” CABIOS (computer applications in the biosciences) (1988) 4:11-17. “Align” is part of the FASTA package version v20u6 (see W. R. Pearson and D. J. Lipman (1988), “Improved Tools for Biological Sequence Analysis”, PNAS 85:2444-2448, and W. R. Pearson (1990) “Rapid and Sensitive Sequence Comparison with FASTP and FASTA,” Methods in Enzymology 183:63-98).

The degree of identity between a sample, or test, sequence of any of the enzyme(s) of the invention and a specified sequence may be determined as follows: The two sequences are aligned using the program “align.” The number of perfect matches (“N-perfect-match”) in the alignment is determined (a perfect match means same amino acid residue in same position of the alignment). The common length of the two aligned sequences is also determined, viz. the total number of amino acids in the alignment (the overlap), including trailing and leading gaps created by the alignment, if any (“N-overlap”). The degree of identity is calculated as the ratio between “N-perfect-match” and “N-overlap” (for conversion to percentage identity, multiply by 100).

The degree of identity between the sample, or test, sequence and a specified sequence may also be determined as follows: The sequences are aligned using the program “align.” The number of perfect matches (“N-perfect-match”) in the alignment is determined (a perfect match means same amino acid residue in same position of the alignment). The length of the sample sequence (the number of amino acid residues) is determined (“N-sample”). The degree of identity is calculated as the ratio between “N-perfect-match” and “N-sample” (for conversion to percentage identity, multiply by 100).

The degree of identity between the sample, or test, sequence and a specified sequence may also be determined as follows: The sequences are aligned using the program “align.” The number of perfect matches (“N-perfect-match”) in the alignment is determined (a perfect match means same amino acid residue in same position of the alignment). The length of the specified sequence (the number of amino acid residues) is determined (“N-specified”). The degree of identity is calculated as the ratio between “N-perfect-match” and “N-specified” (for conversion to percentage identity, multiply by 100).

Preferably, the overlap is at least 20% of the specified sequence (“N-overlap” as defined above, divided by the number of the amino acids in the specified sequence (“N-specified”), and multiplied by 100), more preferably at least 25%, 30%, 35%, 40%, 45%, 50%, 55%, 60%, 65%, 70%, 75%, 80%, 85%, 90%, or at least 95%. This means that at least 20% (preferably 25-95%) of the amino acids of the specified sequence end up being included in the overlap, when the sample sequence is aligned to the specified sequence.

In the alternative, the overlap is at least 20% of the specified sequence (“N-overlap” as defined above, divided by “N-sample” as defined above, and multiplied by 100), more preferably at least 25%, 30%, 35%, 40%, 45%, 50%, 55%, 60%, 65%, 70%, 75%, 80%, 85%, 90%, or at least 95%. This means that at least 20% (preferably 25-95%) of the amino acids of the sample sequence end up being included in the overlap, when aligned against the specified sequence.

The activity of the enzyme(s) of the invention can be measured using any suitable assay. Generally, assay-pH and assay-temperature may be adapted to the enzyme in question. Examples of assay-pH-values are pH 2, 3, 4, 5, 6, 7, 8, 9, 10, 11, or 12. Examples of assay-temperatures are 30, 35, 37, 40, 45, 50, 55, 60, 65, 70, 80, 90, or 95° C. Preferred pH values and temperatures are in the physiological range, such as pH values of 4, 5, 6, 7, or 8, and temperatures of 30, 35, 37, or 40° C.

For example, protease activity can be measured using any assay, in which a substrate is employed, that includes peptide bonds relevant for the specificity of the protease in question.

Examples of suitable enzyme assays are included in the experimental part, see Example 2 in particular. Other examples are the Ph.Eur. assays for lipase and amylase activity.

### Medicament

In the present context, the term “medicament” means a compound, or mixture of compounds, that treats, prevents and/or alleviates the symptoms of disease; preferably that treats and/or alleviates the symptoms of disease. The medicament may be prescribed by a physician, or it may be an over-the-counter product.

### Pharmaceutical Compositions

Isolation, purification, and concentration of the enzyme(s) of the invention may be carried out by conventional means. For example, they may be recovered from a fermentation broth by conventional procedures including, but not limited to, centrifugation, filtration, extraction, spray-drying, evaporation, or precipitation, and further purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulphate precipitation), SDS-PAGE, or extraction (see, e.g., Protein Purification, J.-C. Janson and Lars Ryden, editors, VCH Publishers, New York, 1989).

The preparation of a pure protease of the invention is described in Example 1 herein. In this example, the gene encoding the so-called component C protease (SEQ ID NO: 4, encoded by SEQ ID NO: 3) was deleted from the Bacillus licheniformis production strain by site-directed mutagenesis, as is known in the art. Another approach to the deletion of this gene could be, e.g., classical mutation as described in, e.g., U.S. Pat. No. 4,266,031, preferably combined with state of the art high-throughput screening methods.

In Example 1, the cell expressing the protease of SEQ ID NO: 2 is derived from a wildtype strain of Bacillus licheniformis, viz. strain ATCC 14580, which is publicly available from the American Type Culture Collection, ATCC. It may be preferable to insert one or more additional copies of a gene encoding a protease of the invention, for example a gene encoding amino acids 1-274 of SEQ ID NO: 2, in this cell. This can be done, e.g., as described in WO 02/00907, using, e.g., a promoter disclosed in WO 99/43835.

In a particular embodiment, concentrated solid or liquid preparations of each of the enzyme(s) are prepared separately. These concentrates may also, at least in part, be separately formulated, as explained in more detail below.

In a further particular embodiment, the enzyme(s) are incorporated in the pharmaceutical compositions of the invention in the form of solid concentrates. The enzyme(s) can be brought into the solid state by various methods as is known in the art. For example, the solid state can be either crystalline, where the enzyme molecules are arranged in a highly ordered form, or a precipitate, where the enzyme molecules are arranged in a less ordered, or disordered, form.

Crystallization may, for example, be carried out at a pH close to the pI of the enzyme(s) and at low conductivity, for example 10 mS/cm or less, as described in EP 691982.

Various precipitation methods are known in the art, including precipitation with salts, such as ammonium sulphate, and/or sodium sulphate; with organic solvents, such as ethanol, and/or isopropanol; or with polymers, such as PEG (Poly Ethylene Glycol). In the alternative, the enzyme(s) can be precipitated from a solution by removing the solvent (typically water) by various methods known in the art, e.g. lyophilization, evaporation (for example at reduced pressure), and/or spray drying.

In a further particular embodiment, the solid concentrate of the enzyme(s) has a content of active enzyme protein of at least 50% (w/w) by reference to the total protein content of the solid concentrate. In still further particular embodiments, the content of active enzyme protein, relative to the total protein content of the solid concentrate is at least 55, 60, 65, 70, 75, 80, 85, 90, or at least 95% (w/w). The protein content can be measured as is known in the art, for example by densitometer scanning of coomassie-stained SDS-PAGE gels, by using a commercial kit, such as Protein Assay ESL, order no. 1767003, which is commercially available from Roche, or on the basis of the method described in Example 8 of WO 01/58276.

Preferably, the protease enzyme protein constitutes at least 50%, more preferably at least 55, 60, 65, 70, 75, 80, 85, 90, 92, 94, 95, 96, or at least 97% of the protein spectrum of the solid protease concentrate for use according to the invention, as measured by densitometer scanning of a coomassie-stained SDS-PAGE gel.

A pharmaceutical composition of the invention comprises the enzyme(s), preferably in the form of concentrated enzyme preparations, more preferably solid concentrates, together with at least one pharmaceutically acceptable auxiliary, or subsidiary, material such as (i) at least one carrier and/or excipient; or (ii) at least one carrier, excipient, diluent, and/or adjuvant. Non-limiting examples of, optional, other ingredients, all pharmaceutically acceptable, are disintegrators, lubricants, buffering agents, moisturizing agents, preservatives, flavouring agents, solvents, solubilizing agents, suspending agents, emulsifiers, stabilizers, propellants, and vehicles.

Generally, depending i.a. on the medical indication in question, the composition of the invention may be designed for all manners of administration known in the art, preferably including enteral administration (through the alimentary canal). Thus, the composition may be in solid, semi-solid, liquid, or gaseous form, such as tablets, capsules, powders, granules, microspheres, ointments, creams, foams, solutions, suppositories, injections, inhalants, gels, microspheres, lotions, and aerosols. The medical practitioner will know to select the most suitable route of administration and of course avoid potentially dangerous or otherwise disadvantageous administration routes.

The following methods and auxiliary materials are therefore also merely exemplary and are in no way limiting.

For solid oral preparations, the enzyme(s) can be used alone or in combination with appropriate additives to make pellets, micropellets, tablets, microtablets, powders, granules or capsules, for example, with conventional carriers, such as lactose, mannitol, corn starch, or potato starch; with excipients or binders, such as crystalline, or microcrystalline, cellulose, cellulose derivatives, acacia, corn starch, or gelatins; with disintegrators, such as corn starch, potato starch, or sodium carboxymethylcellulose; with lubricants, such as carnauba wax, white wax, shellac, waterless colloid silica, polyethylene glycol (PEGs, also known under the term macrogol) from 1500 to 20000, in particular PEG 4000, PEG 6000, PEG 8000, povidone, talc, monolein, or magnesium stearate; and if desired, with diluents, adjuvants, buffering agents, moistening agents, preservatives such as methylparahydroxybenzoate (E218), colouring agents such as titanium dioxide (E171), and flavouring agents such as saccharose, saccharin, orange oil, lemon oil, and vanillin. Oral preparations are examples of preferred preparations for treatment of the medical indication of PEI.

The enzyme(s) can also, quite generally, be formulated into liquid oral preparations, by dissolving, suspending, or emulsifying them in an aqueous solvent such as water, or in non-aqueous solvents such as vegetable or other similar oils, synthetic aliphatic acid glycerides, esters of higher aliphatic acids, propylene glycol, polyethylene glycol such as PEG 4000, or lower alcohols such as linear or ramified C1-C4 alcohols, for example 2-propanol; and if desired, with conventional subsidiary materials or additives such as solubilizers, adjuvants, diluents, isotonic agents, suspending agents, emulsifying agents, stabilizers, and preservatives.

Furthermore, the enzyme(s) can generally be made into suppositories for rectal administration by mixing with a variety of bases such as emulsifying bases or water-soluble bases. The suppository can include vehicles such as cocoa butter, carbowaxes and polyethylene glycols, which melt at body temperature, yet are solidified at room temperature.

The use of liposomes as a delivery vehicle is another method of possible general interest. The lipids may be any useful combination of known liposome forming lipids, including cationic or zwitterionic lipids, such as phosphatidylcholine. The remaining lipid will normally be neutral or acidic lipids, such as cholesterol, phosphatidyl serine, phosphatidyl glycerol, and the like. For preparing the liposomes, the procedure described by Kato et al. (1991) J. Biol. Chem. 266:3361 may be used.

Unit dosage forms for oral or rectal administration such as syrups, elixirs, powders, and suspensions may be provided wherein each dosage unit, for example, teaspoonful, tablespoonful, capsule, tablet or suppository, contains a predetermined amount of the enzyme(s). Similarly, unit dosage forms for injection may comprise the enzyme(s) in a composition as a solution in sterile water, normal saline, or another pharmaceutically acceptable carrier.

The term “unit dosage form”, as used herein, refers to physically discrete units suitable as unitary dosages for human and animal subjects, each unit containing a predetermined quantity of enzyme(s) in an amount sufficient to produce the desired effect.

In a particular embodiment, the pharmaceutical composition of the invention is for enteral, preferably oral, administration.

In further particular embodiments, the oral composition is (i) a liquid composition containing crystals of the enzyme(s); (ii) a liquid suspension of sediments of (highly) purified enzyme(s); (iii) a gel containing the enzyme(s) in solid or solubilized form; (iv) a liquid suspension of immobilized enzyme(s) or of enzymes adsorbed to particles and the like; or (v) a solid composition in the form of enzyme(s)-containing powder, pellets, granules, or microspheres, if desired in the form of tablets, capsules, or the like, that are optionally coated, for example with an acid-stable coating.

In another particular embodiment of the composition, the enzyme(s) are compartmentalized, viz. separated from each other, for example by means of separate coatings.

In a still further particular embodiment of the composition, the protease is separated from other enzyme components of the composition, such as the lipase, and/or the amylase.

The dosage of the enzyme(s) will vary widely, depending on the specific enzyme(s) to be administered, the frequency of administration, the manner of administration, the severity of the symptoms, and the susceptibility of the subject to side effects, and the like. Some of the specific enzymes may be more potent than others.

Examples of solid oral preparations of the enzyme(s) of the invention comprise: (i) a protease of the invention comprising an amino acid sequence which has at least 50% identity to amino acids 1-274 of SEQ ID NO: 2; (ii) a lipase having at least 70% identity to a lipase having amino acids 1-269 of SEQ ID NO: 15: and (iii) an amylase having at least 70% identity to an amylase selected from the group consisting of a) an amylase having amino acids 1-481 of SEQ ID NO: 16, b) an amylase having amino acids 1-481 of SEQ ID NO: 17, and c) an amylase having amino acids 1-483 of SEQ ID NO: 18; wherein preferably the anticipated daily clinical dosages of the enzymes of (i), (ii), and (iii) are as follows (all in mg enzyme protein per kg of bodyweight (bw)): For the protease of (i): 0.005-500, 0.01-250, 0.05-100, or 0.1-50 mg/kg bw; for the lipase of (ii): 0.01-1000, 0.05-500, 0.1-250, or 0.5-100 mg/kg bw; for the amylase of (iii): 0.001-250, 0.005-100, 0.01-50, or 0.05-10 mg/kg bw.

A preferred example of solid oral preparations of the enzyme(s) of the invention comprises: (i) a protease comprising, preferably having, amino acids 1-274 of SEQ ID NO: 2; (ii) a lipase comprising amino acids 2-269 of SEQ ID NO: 15, and/or (iii) an amylase comprising amino acids 1-481 of SEQ ID NO: 16.

Examples of anticipated daily clinical dosages of the enzymes of (i), (ii), and (iii) are as follows (all in mg enzyme protein per kg of bodyweight (bw)): For the protease of (i): 0.05-100, 0.1-50, or 0.5-25 mg/kg bw; for the lipase of (ii): 0.1-250, 0.5-100, or 1-50 mg/kg bw; for the amylase of (iii): 0.01-50, 0.05-10, or 0.1-5 mg/kg bw.

The amide (peptide) bonds, as well as the amino and carboxy termini, may be modified for greater stability on oral administration. For example, the carboxy terminus may be amidated.

Particular embodiments of pharmaceutical compositions of the invention, suitable for the treatment of digestive disorders, PEI, pancreatitis, cystic fibrosis, diabetes type I, and/or diabetes type II, may be prepared by incorporating the enzyme(s) of the invention into pellets. The pellets may generally comprise from 10-90% (w/w, relative to the dry weight of the resulting pellets) of a physiologically acceptable organic polymer, from 10-90% (w/w, relative to the dry weight of the resulting pellets) of cellulose or a cellulose derivative, and from 80-20% (w/w, relative to the dry weight of the resulting pellets) of the enzyme(s), the total amount of organic polymer, cellulose or cellulose derivative and enzyme(s) making up to 100% in each case.

The physiologically acceptable organic polymer can be selected from the group consisting of polyethylene glycol 1500, polyethylene glycol 2000, polyethylene glycol 3000, polyethylene glycol 4000, polyethylene glycol 6000, polyethylene glycol 8000, polyethylene glycol 10000, polyethylene glycol 20000, hydroxypropyl methylcellulose, polyoxyethylen, copolymers of polyoxyethylene-polyoxypropylen and mixtures of said organic polymers. Polyethylene glycol 4000 is preferred as physiologically acceptable organic polymer.

The cellulose or a cellulose derivative can e.g. be selected from cellulose, cellulose acetate, cellulose fatty acid ester, cellulose nitrates, cellulose ether, carboxymethyl cellulose, ethyl cellulose, hydroxyethyl cellulose, hydroxypropyl cellulose, methyl cellulose, methyl ethylcellulose and methylhydroxypropyl cellulose. Cellulose, in particular microcrystalline cellulose is preferred as cellulose or cellulose derivative.

The resulting pellets may be coated with a suitable enteric coating, other non functional coating or be used directly without such coating. Further, the resulting pellets may be filled in capsules like hard gelatin capsules or gelatin free capsules of a suitable size for therapy of a disorder or disease as described in more detail above. In an embodiment of the invention, pellets produced from different enzyme types, in particular from lipase, protease and/or amylase may be filled into said capsules. While filling the capsules with the different enzyme types, the dosing of the single enzyme types (viz. lipase, protease or amylase) may be adapted to specific needs of a certain indication group or a certain patient subgroup by adding a specified amount of any of lipase, protease and/or amylase to the capsules, i.e. capsules may be produced which vary in their specific ratios of lipase:protease:amylase.

Preferred pharmaceutical compositions of the lipase of the invention are described in WO 2005/092370, in particular formulations comprising the preferred excipients mentioned therein. In a particularly preferred embodiment, the pharmaceutical composition comprises a macrogolglyceride mixture of mono-, di- and tri-acylglycerides and polyethylene glycol (PEG) mono- and di-esters of aliphatic C6-C22 carboxylic acids, and also possibly small proportions of glycerol and free polyethylene glycol.

The polyethylene glycol (PEG) contained in the macrogolglyceride mixtures is preferably PEG which has on average 6 to at most 40 ethylene oxide units per molecule or a molecular weight of between 200 and 2000.

One further aspect of the invention provides for the pharmaceutical composition of the enzyme(s) of the invention to comprise a system consisting of surfactant, co-surfactant and lipophilic phase, the system having an HLB value (Hydrophilic-Lipophilic Balance) greater than or equal to 10 and a melting point greater than or equal to 30° C. In a preferred embodiment, the system has an HLB value of 10 to 16, preferably of 12 to 15, and has a melting point of between 30 and 600° C., preferably between 40 and 500° C. In particular, the system characterised by HLB value and melting point is a mixture of mono-, di- and triacylgylcerides and mono- and diesters of polyethylene glycol (PEG) with aliphatic carboxylic acids with 8 to 20, preferably 8 to 18, carbon atoms, whereby the polyethylene glycol preferably has about 6 to about 32 ethylene oxide units per molecule, and the system optionally contains free glycerin and/or free polyethylene glycol. The HLB value of such a system is preferably regulated by the chain length of the PEG. The melting point of such a system is regulated by the chain length of the fatty acids, the chain length of the PEG and the degree of saturation of the fatty-acid chains, and hence the starting oil for the preparation of the macrogolglyceride mixture.

“Aliphatic C8-C18 carboxylic acids” designates mixtures in which caprylic acid (C8), capric acid (C10), lauric acid (C12), myristic acid (C14), palmitic acid (C16) and stearic acid (C18) are contained in a significant and variable proportion, if these acids are saturated, and the corresponding unsaturated C8-C18 carboxylic acids. The proportions of these fatty acids may vary according to the starting oils.

Such a mixture of mono-, di- and triacylgylcerides and mono- and diesters of polyethylene glycol (PEG) with aliphatic carboxylic acids with 8 to 18 carbon atoms can for example be obtained by a reaction between a polyethylene glycol with a molecular weight of between 200 and 1500 and a starting oil, the starting oil consisting of a triglyceride mixture with fatty acids which are selected from the group containing caprylic acid, capric acid, lauric acid, myristic acid, palmitic acid, stearic acid, oleic acid and linolenic acid, individually or as a mixture. Optionally, the product of such a reaction may also contain small proportions of glycerine and free polyethylene glycol.

Such mixtures are commercially available for example under the trade name Gelucire®. One advantageous embodiment of the invention provides that, of the products known under the trade name Gelucire®, in particular “Gelucire® 50/13” and/or “Gelucire® 44/14” represent suitable mixtures for use in the pharmaceutical preparations according to the invention.

Gelucire® 50/13 is a mixture with mono-, di- and triacylglycerides and mono- and diesters of polyethylene glycol, with palmitic acid (C16) and stearic acid (C18) at 40% to 50% and 48% to 58%, respectively making up the major proportion of bound fatty acids. The proportion of caprylic acid (C8) and capric acid (C10) is less than 3% in each case, and the proportion of lauric acid (C12) and myristic acid (C14) in each case is less than 5%.

Gelucire® 44/14 is a mixture with mono-, di- and triacylgylcerides and mono- and diesters of polyethylene glycol, the respective proportions of palmitic acid (C16) being 4 to 25%, stearic acid (C18) 5 to 35%, caprylic acid (C8) less than 15%, capric acid (C10) less than 12%, lauric acid (C12) 30 to 50% and myristic acid (C14) 5 to 25%. Gelucire® 44/14 can for example be prepared by an alcoholysis/esterification reaction using palm kernel oil and polyethylene glycol 1500.

A preferred embodiment of the present invention provides for a pharmaceutical composition of the enzyme(s) of the invention which comprises a system containing a mixture of mono-, di- and triacyl-glycerides and polyethylene glycol mono- and diesters of aliphatic C8-C18 carboxylic acids and also possibly small proportions of glycerin and free polyethylene glycol, the system having a melting point between 40° C. and 55° C. and an HLB value in the range between 12 and 15. More preferred, the system has a melting point between 44° C. and 50° C. and an HLB value in the range from 13-14. Alternatively, the system has a melting point around 44° C. and an HLB value of 14, or the system has a melting point around 50° C. and an HLB value of 13.

### Methods of Treatment

The protease of the invention, optionally in combination with a lipase, and/or an amylase (the enzyme(s) of the invention), is useful in the therapeutic, and/or prophylactic, treatment of various diseases or disorders in animals. The term “animal” includes all animals, and in particular human beings. Examples of animals are non-ruminants, and ruminants, such as sheep, goat, and cattle, e.g. beef cattle, and cow. In a particular embodiment, the animal is a non-ruminant animal. Non-ruminant animals include mono-gastric animals, e.g. horse, pig (including, but not limited to, piglets, growing pigs, and sows); poultry such as turkeys, ducks and chicken (including but not limited to broiler chicks, layers); young calves; pets such as cat, and dog; and fish (including but not limited to salmon, trout, tilapia, catfish and carps; and crustaceans (including but not limited to shrimps and prawns). In a particular embodiment the animal is a mammal, more in particular a human being.

For example, the enzyme(s) are useful in the treatment of digestive disorders like maldigestion or dyspepsia that are often caused by a deficient production and/or secretion into the gastrointestinal tract of digestive enzymes normally secreted from, i.a., the stomach, and the pancreas.

Further, the enzyme(s) are particularly useful in the treatment of PEI. PEI can be verified using, i.a., the Borgström test (JOP. J Pancreas (Online) 2002; 3(5):116-125), and it may be caused by diseases and conditions such as pancreatic cancer, pancreatic and/or gastric surgery, e.g. total or partial resection of the pancreas, gastrectomy, post gastrointestinal bypass surgery (e.g. Billroth II gastroenterostomy); chronic pancreatitis; Shwachman Diamond Syndrome; ductal obstruction of the pancreas or common bile duct (e.g. from neoplasm); and/or cystic fibrosis (an inherited disease in which a thick mucus blocks the ducts of the pancreas). The enzyme(s) may also be useful in the treatment of acute pancreatitis.

The effect of the enzyme(s) on digestive disorders can be measured as generally described in EP 0600868, in which Example 2 describes an in vitro digestibility test for measuring lipase stability test under gastric conditions, and Example 3 an in vitro digestibility test for lipase activity in the presence of bile salts. Corresponding tests can be set up for the protease and amylase. Also WO 02/060474 discloses suitable tests, for example (1) an in vitro test for measuring lipid digestion in a swine test feed, and (2) an in vivo trial with pancreas insufficient swine in which the digestibility of fat, protein and starch is measured.

In a particular embodiment, the effect of the protease of the invention is measured using the in vivo screening test for protease efficacy of Example 3.

As another example, the enzyme(s) are useful in the treatment of Diabetes mellitus type I, and/or type II, in particular for adjuvant treatment in a diabetes therapy of digestive disorders usually accompanying this disease, with a view to diminishing late complications.

The effect on Diabetes mellitus of the enzyme(s) may be determined by one or more of the methods described in WO 00/54799, for example by controlling the level of glycosylated haemoglobin, the blood glucose level, hypoglycaemic attacks, the status of fat-soluble vitamins like vitamins A, D and E, the required daily dosage of insulin, the body-weight index, and hyper glycaemic periods.

In a particular embodiment, the protease of the invention is not for use as a debridement agent, and/or not for use in would healing.

The invention described and claimed herein is not to be limited in scope by the specific embodiments herein disclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

Various references are cited herein, the disclosures of which are incorporated by reference in their entireties.

### EXAMPLES

### Example 1

### Preparation of Purified Bacillus licheniformis Protease

A pure preparation of the Bacillus licheniformis protease of amino acids 1-274 of SEQ ID NO: 1 was prepared as follows:

### Materials and Methods:

TY broth: Tryptone 20 g/l, Yeast extract 5 g/l, FeCl₂, 4H₂O 7 mg/l, MnCl₂, 4H₂O 1 mg/l, MgSO₄, 7H₂O 15 mg/l, pH 7.3.

PS-1 broth: Sucrose 100 g, Soybean meal 40 g, Na₂HPO₄.12H₂O (Merck 6579) 10 g, CaCO₃ 5 g, Pluronic PE 6100 (BASF) 0.1 ml, tap water ad 1000 ml.

### Fermentation:

A strain derived from Bacillus licheniformis ATCC 14580 by deletion of the gene (SEQ ID NO: 3) encoding another protease, was propagated overnight at 37° C. on TY agar medium (TY broth solidified with 2% agar) and inoculated into shake flasks containing 100 ml PS-1 broth. The shake flasks were incubated at 37° C. for 90 hours with a shaking speed of 225 rpm.

### Purification:

The fermentation broth was flocculated and the cells were separated from the enzyme-containing liquid by centrifugation. SDS polyacrylamide gel electrophoresis of the supernatant revealed a strong band of a relative molecular weight of approximately 31 kDa corresponding to the desired protease. The presence of a strong protease activity in the supernatant was also confirmed by the presence of large clearing zones on 1% skim milk agar plates, pH 7 and 9. As a next step, the liquid from the centrifuge was polish filtered to remove remaining suspended solids and then concentrated by ultrafiltration using appropriate membranes i.e. with a cut-off value below the size of the protease. Finally the concentrate was germ-filtered.

100 ml of the germ-filtered liquid concentrate was diluted 10× in 100 mM H₃BO₃, 10 mM succinic acid/NaOH, 2 mM CaCl₂, pH 7.0. The pH of the resulting protease solution was 7.0. 120 ml thereof was applied to a 100 ml bacitracin-agarose column (UpFront Chromatography, catalogue no. 600-0100) equilibrated in 100 mM H₃BO₃, 10 mM succinic acid/NaOH, 2 mM CaCl₂, pH 7.0. After a thorough wash of the column with the equilibration buffer, the column was step-eluted with 100 mM H₃BO₃, 10 mM succinic acid/NaOH, 2 mM CaCl₂, 1M NaCl, pH 7.0, 25% (v/v) isopropanol. The Bacitracin-silica step was repeated 7 times (8 times in total). All the eluates were combined (420 ml) and the eluates were diluted to 15 L with demineralized water. The pH of the diluted protease was adjusted to pH 6.0 with 20% CH₃COOH and applied to a 400 ml SP-sepharose FF column equilibrated in 50 mM H₃BO₃, 5 mM succinic acid/NaOH, 1 mM CaCl₂, pH 6.0. The column was washed thoroughly with the equilibration buffer and the column was eluted with a linear NaCl gradient (0-0.5M) over 3 column volumes. The eluted protease peak (200 ml) was transferred to 20 mM HEPES/NaOH, 100 mM NaCl, 1 mM CaCl₂, pH 7.0 by buffer exchange on a 1.4 L G25 sephadex column (HEPES is 4-(2-hydroxyethyl)-1-piperazineethanesulfonic acid). The buffer exchanged protease (340 ml) was filtered on a 0.22μ filtration unit (such as Corning, catalogue no. 431097).

### Example 2

### Enzyme Assays

### Protease Suc-AAPF-pNA Assay

### Substrate: Suc-AAPF-pNA (Sigma® S-7388).

Assay buffer: 100 mM succinic acid, 100 mM HEPES (Sigma H-3375), 100 mM CHES (Sigma C-2885), 100 mM CABS (Sigma C-5580), 1 mM CaCl₂, 150 mM KCl, 0.01% Triton® X-100 adjusted to pH 9.0 with HCl or NaOH.

### Assay Temperature: 25° C.

300 μl diluted protease sample was mixed with 1.5 ml of the assay buffer and the activity reaction was started by adding 1.5 ml pNA substrate (50 mg dissolved in 1.0 ml DMSO and further diluted 45× with 0.01% Triton® X-100) and, after mixing, the increase in A405 was monitored by a spectrophotometer as a measurement of the protease activity. The protease samples were diluted prior to the activity measurement in order to ensure that all activity measurements fell within the linear part of the dose-response curve for the assay.

### Protease FIP Assay

Protease activity may also be determined using a FIP assay (Fédération Internationale Pharmaceutique), 1 FIP-unit=1 Ph.Eur.-unit (European Pharmacopoeia). This assay is described, together with other FIP assays in: Fédération Internationale Pharmaceutique, Scientific Section International Commission for the standardisation of pharmaceutical enzymes. a) “Pharmaceutical Enzymes,” Editors: R. Ruyssen and A. Lauwers, E. Story Scientia, Ghent, Belgium (1978), b) European Pharmacopoeia. See also Deemester et al in Lauwers A, Scharpé S (eds): Pharmaceutical Enzymes, New York, Marcel Dekker, 1997, p. 343-385. This assay was used for determining protease activity in pancreatin. For determining FIP activity of microbial proteases, the activation step by adding enterokinase was omitted.

Principle: The substrate casein is hydrolysed by protease at pH 7.5 and at a temperature of 35° C. The reaction is stopped by addition of trichloroacetic acid, and non-degraded casein is filtered off. The quantity of peptides remaining in solution is determined by spectrophotometry at 275 nm.

Definition of the activity: The protease activity is determined as the quantity of peptides not precipitated by a 5.0% (wt/vol, i.e. 5.0 g/100 ml) solution of trichloroacetic acid, by reference to a pancreas reference powder (protease reference standard) of known FIP activity.

### Materials and Methods:

Casein Solution:

1.25 g casein (dry matter), e.g. Calbiochem no. 218680, is suspended in water until a practically clear solution is obtained. pH is adjusted to 8.0, and the solution is diluted with water to a final volume of 100 ml. Here and in the following, water means deionized water.

Borate Buffer pH 7.5:

2.5 g sodium chloride, 2.85 g disodium tetraborate and 10.5 g boric acid are dissolved in 900 ml water, pH is adjusted to pH 7.5+/−0.1 and diluted to 1000 ml with water.

Filter Paper:

Folded filters with a diameter of 125 mm, e.g. Schleicher & Schuell no. 1574½. Test of filter paper: Filter 5 ml of 5.0% trichloroacetic acid through the filter. The absorption at 275 nm of the filtrate should be less than 0.04, using unfiltered trichloroacetic acid solution as a blank.

Protease Reference Standard:

Protease (pancreas) commercially available from the International Commission on Pharmaceutical Enzymes, Centre for Standards, Harelbekestraat 72, B-9000 Ghent, Belgium. The standard has a labelled activity (A) in FIP/Ph.Eur.-units/g. Accurately weigh a quantity corresponding to approx. 130 protease-FIP/Ph.Eur.-units. Add a spatula tip of sea sand, wet with a few drops of ice-cold 0.02M calcium chloride (pH 6.0-6.2), and triturate the whole with a flat-ended glass rod. Dilute with approx. 90 ml of the same ice-cold calcium chloride solution and stir the suspension for 15 to 30 minutes in an ice-bath. pH is adjusted to 6.1 and the volume is adjusted to 100 ml with the same calcium chloride solution. 5.0 ml of this suspension is diluted with borate buffer pH 7.5 to 100 ml. For the activity test, 1.0, 2.0 and 3.0 ml of this solution is used as reference (in what follows designated S1, S2, and S3, S for Standard).

Test Suspension:

Prepare a suspension of the sample as described above for the protease reference standard, using a sample amount equivalent to approx. 260 FIP/Ph.Eur.-units. pH is adjusted to 6.1 and water is added to 100 ml. 5.0 ml of this solution is mixed with 5 ml of calcium chloride solution. 5 ml of this dilution is further diluted to 100 ml with borate buffer. Use 2.0 ml of this solution for the assay (in what follows the sample is designated Un, sample of unknown activity, number n).

Assay Procedure (Activity Test):

The assay is performed for the three reference suspensions (S1, S2, S3) and for the sample suspension (Un), all in triplicate. One blank per sample is sufficient (designated S1b, S2b, S3b, and Unb, respectively). A blind (B) is prepared without sample/standard as compensation liquid for the spectrophotometer. Borate buffer is added to tubes as follows: Blind (B) 3.0 ml; sample (Un) 1.0 ml; standards (S1, S2 and S3) 2.0, 1.0 and 3.0 ml, respectively. Protease reference standard is added to S1, S2 and S3 as follows: 1.0, 2.0, and 3.0 ml, respectively. The test suspension is added to the sample tubes as follows (Un): 2.0 ml. 5 ml trichloroacetic acid is added to all blinds (S1b, S2b, S3b, Unb and B) followed by immediate mixing. All tubes are stopped with a glass stopper and placed together with the substrate solution in a water-bath at constant temperature (35+/−0.5° C.). When temperature equilibration is reached, at time zero, 2.0 ml casein solution is added to tubes S1, S2, S3 and Un, followed by immediate mixing. Exactly 30 minutes after, 5.0 ml. trichloroacetic acid is added to each of tubes S1, S2, S3 and Un, followed by immediate mixing. The tubes are withdrawn from the water bath and allowed to stand at room temperature for 20 minutes to complete the precipitation of the proteins. The content of each tube is filtered twice through the same filter, and the absorption of the filtrates is measured at 275 nm using the filtrate from tube B as compensation liquid. The activity of the sample (Un) in FIP units is calculated relative to the known labelled activity (A) of the standards (S1, S2, S3). The absorption values minus the respective blinds (e.g. the absorption of S1 minus the absorption of S1b) should lie in the interval of 0.15-0.60.

### Protease AU Assay

Denatured haemoglobin (0.65% (w/w) in urea-containing 6.7 mM KH₂PO₄/NaOH buffer, pH 7.50) is degraded at 25° C. for 10 minutes by the protease and un-degraded haemoglobin is precipitated with trichloroacetic acid (TCA) and removed by filtration. The TCA-soluble haemoglobin degradation products in the filtrate are determined with Folin & Ciocalteu's phenol reagent (1 volume of Folin-Ciocalteu Phenol Reagent Merck 9001.0500 to 2 volumes of demineralised water), which gives a blue colour with several amino acids (being measured at 750 nm). The activity unit (AU) is measured and defined by reference to a standard. The denatured haemoglobin substrate may be prepared as follows: 1154 g urea (Harnstoff, Merck 8487) is dissolved in 1000 ml demineralised water, 240.3 g NaOH is added and then, slowly, 63.45 g haemoglobin (Merck 4300) is added, followed by 315.6 g KH₂PO₄, and demineralised water ad 3260 g. pH is adjusted to 7.63. More details and a suitable Alcalase standard are available on request from Novozymes A/S, Krogshoejvej 36, DK-2880 Bagsvaerd, Denmark (assay no. EB-SM-0349.01).

### Lipase pNP Assay

Substrate: para-Nitro-Phenyl (pNP) Valerate

### Assay pH: 7.7

Assay temperature: 40° C.
Reaction time: 25 min

The digested product with yellow colour has a characteristic absorbance at 405 nm. Its quantity is determined by spectrophotometry. One lipase unit is the amount of enzyme which releases 1 micromole titratable butyric acid per minute under the given assay conditions. A more detailed assay description, AF95/6-GB, is available on request from Novozymes A/S, Krogshoejvej 36, DK-2880 Bagsvaerd, Denmark.

### Lipase LU Assay

In this assay, the lipase-catalysed degradation of 0.16M tributyrin (glycerol tributyrate, Merck 1.01958.000) at pH 7.00 and 30° C. (+/−1° C.) is followed by pH-stat titration of released butyric acid with 0.025 M de-gassed, CO₂-free sodium hydroxide (Sodium hydroxide titrisol, Merck 9956). The consumption of the titrant is recorded as a function of time.

The substrate is emulsified with a 0.6% w/v Gum arabic emulsifier (20.0 g Gum Arabic, 89.5 g NaCl, 2.05 g KH₂PO₄, add water to 1.5 l, leave until completely dissolved, add 2700 ml glycerol, adjust pH to 4.5. 90 ml of tributyrin is mixed with 300 ml gum arabic emulsifier and 1410 ml demineralised water and homogenised for 3 minutes using e.g. a Silverson emulsifier L4RT at 7000 rpm and then adjusted to pH 4.75). Lipase-samples are diluted first in 0.1M glycine buffer pH 10.8, next in demineralized water, aiming at an activity level of 1.5-4.0 LU/ml. 15 ml of the emulsified substrate solution is poured into the titration vessel. 1.0 ml sample solution is added, and pH is maintained at 7.0 during the titration. The amount of titrant added per minute to maintain a constant pH is measured. The activity calculation is based on the mean slope of the linear range of the titration curve. A standard of known activity may be used as a level check.

1 LU (lipase unit) is the amount of enzyme which releases 1 micro mole titratable butyric acid per minute under the assay conditions given above. 1 kLU (kilo Lipase Unit)=1000 LU.

A more detailed assay description, EB-SM-0095.02, is available on request from Novozymes A/S, Krogshoejvej 36, DK-2880 Bagsvaerd, Denmark.

### Lipase pH Stat Assay

This assay is based on the lipase-catalysed release of fatty acids from an olive oil emulsion in the presence of 0.65 mM bile salts. The substrate is emulsified with gum arabic as emulsifier (175 g olive oil emulsified with 630 ml gum arabic solution (474.6 g gum arabic, 64 g calcium chloride in 4000 ml water) for 15 min in a blender; after cooling to room temperature, pH is adjusted to pH 6.8-7.0 using 4 M NaOH).

For the determination, 19 ml of the emulsion and 10 ml bile salts solution (492 mg bile salts are dissolved in water and filled up to 500 ml) are mixed in the reaction vessel and heated to 36.9° C. to 37.5° C. Reaction is started by addition of 1.0 ml of enzyme solution. The released acid is titrated automatically at pH 7.0 by addition of 0.1 M sodium hydroxide for a total of 5 min. The activity is calculated from the slope of the titration curve between the 1st and the 5th minute. For calibration, a standard is measured at three different levels of activity.

### Amylase

Substrate: Phadebas tablets (Pharmacia Diagnostics; cross-linked, insoluble, blue-coloured starch polymer, which is mixed with bovine serum albumin and a buffer substance, and manufactured into tablets)

### Assay Temperature: 37° C.

Assay pH: 4.3 (or 7.0, if desired)
Reaction time: 20 min

After suspension in water the starch is hydrolyzed by the alpha-amylase, giving soluble blue fragments. The absorbance of the resulting blue solution, measured at 620 nm, is a function of the alpha-amylase activity. One Fungal alpha-Amylase Unit (1 FAU) is the amount of enzyme which breaks down 5.26 g starch (Merck, Amylum solubile Erg. B. 6, Batch 9947275) per hour at the standard assay conditions. A more detailed assay description, APTSMYQI-3207, is available on request from Novozymes A/S, Krogshoejvej 36, DK-2880 Bagsvaerd, Denmark.

### Example 3

### In Vivo Screening Test of the Bacillus licheniformis Protease

The purified Bacillus licheniformis protease of Example 1 was tested in female Göttingen minipigs (Ellegaard) with pancreatin as a benchmark. Pancreatic Exocrine Insufficiency (PEI) was induced in the minipigs by ligation of the pancreatic duct, and they were also fitted with an ileo-caecal re-entrant cannula, all under halothane anaesthesia and at a weight of about 25 kg, as described in Tabeling et al., J. 1999, Studies on nutrient digestibilities (pre-caecal and total) in pancreatic duct-ligated pigs and the effects of enzyme substitution, J. Anim. Physiol. A. Anim. Nutr. 82: 251-263 (hereinafter referred to as “Tabeling 1999”); and in Gregory et al., J. 1999. Growth and digestion in pancreatic duct ligated pigs, Effect of enzyme supplementation in “Biology of the Pancreas in Growing Animals” (SG Pierzynowski & R. Zabielski eds), Elsevier Science BV, Amsterdam, pp 381-393 (hereinafter referred to as “Gregory et al 1999”). A period of at least 4 weeks was allowed for recovery from surgery, before studies were commenced. Prior to study begin, the PEI status of each pig was confirmed via the stool chymotrypsin test (commercially available from Immundiagnostik AG, Wiesenstrasse 4, D-64625 Bensheim, Germany, with catalogue No. K 6990).

During the studies, the pigs were housed in modified metabolism cages on a 12:12 h light-dark cycle and allowed free access to water and fed two meals/day. To assess protease efficacy, the pigs were fed a 250 g test meal mixed with 1 liter of water, 0.625 g Cr₂O₃ (chromic oxide marker) and into which differing amounts of protease (0, 1000, 2500, 6000 FIP U protease/meal (protease FIP units, see Example 2)) were mixed immediately before feeding. The test meal contained 21.4% protein, 51.9% starch, 2.6% fat, and had the following composition (g/100 g dry matter): Fish meal 3.5, poultry meat meal 10.2, wheat flour 29.5, shelled rice 14, potato starch 11, maize starch 14, casein 5.9, cellulose powder 4.3, vitamins, minerals and trace elements 7.6 (as per the nutritional requirement for pigs, see e.g. Table A of WO 01/58276).

Ileal chyme was collected on ice for a total of 8 h after first appearance of the meal marker in the ileum (green chyme) and stored at −20° C. before analysis. At least one day washout was allowed between separate determinations.

In brief, the frozen samples were freeze-dried and analysed for dry matter (DM) and crude protein. DM was estimated by weight after freeze-drying followed by 8 h incubation at 103° C. Crude protein was calculated as nitrogen (N) multiplied by a factor 6.25, i.e. Crude protein (g/kg)=N (g/kg)×6.25 as stated in Animal Nutrition, 4th edition, Chapter 13 (Eds. P. McDonald, R. A. Edwards and J. F. D. Greenhalgh, Longman Scientific and Technical, 1988, ISBN 0-582-40903-9). The nitrogen content was determined by the Kjeldahl method (Naumann and Bassler, 1993, Die chemische Untersuchung von Futtermitteln. 3 edition VDLUFA-Verlag, Darmstadt, Germany (VDLUFA=Verband Deutscher Landwirtschaftlicher Untersuchungs-und Forschungsanstalten).

Calculation of apparent pre-caecal protein digestibility was made according to the formula:

$$Apparent digestibility ( \% ) = 100 - [ \frac{\% {Cr}_{2} {O}_{3} in feed \cdot \% protein in sample}{\% {Cr}_{2} {O}_{3} in sample \% protein in feed} \cdot 100 ]$$

in which Cr₂O₃ and protein were expressed as g/100 g dry matter. The amount of Cr₂O₃ can be determined by methods known in the art, preferably by oxidation to chromate and measurement of extinction at 365 nm, as described by Petry and Rapp in Zeitung für Tierphysiologie (1970), vol. 27, p. 181-189. The results of this study are depicted in Table 1.

TABLE 1
Influence of enzyme supplementation on apparent protein digestibility

| Enzyme Supplement | 0 | 1000 FIP U | 2500 FIP U | 6000 FIP U |
|---|---|---|---|---|
| No supplement | 14.7 +/− 2.1 | | | |
| Pancreatin | | 31.7 +/− 12.4 | 59.4 +/− 4.9 | 70.7 +/− 0.9 |
| Bacillus licheniformis | | 39.1 +/− 8.6 | 58.5 +/− 11.3 | 65.5 +/− 1.1 |
| protease | | | | |

Values are mean ± SD

From the results in Table 1 it is apparent that the protease of SEQ ID NO: 2 according to the invention performs with the same activity as known pancreatin preparations. The protease of the invention caused a strong and dose dependent improvement on protein digestibility, already showing a highly efficient improvement at the lowest dosage tested.

### Example 4

### In Vitro Testing of Proteases

Various proteases were tested in vitro for their ability to degrade protein under digestion-simulating conditions.

### Proteases

The following subtilisin proteases of the invention were tested: The Bacillus licheniformis protease of amino acids 1-274 of SEQ ID NO: 1; the Bacillus amyloliquefaciens protease of amino acids 1-275 of SEQ ID NO: 10, and variant 99aE of the Bacillus lentus protease of amino acids 1-269 of SEQ ID NO: 11 (an E (Glu) being inserted after amino acid residue no. 99, S (Ser), in amino acids 1-269 of SEQ ID NO: 11). These proteases all have a percentage identity to amino acids 1-274 of SEQ ID NO: 1 of above 50%.

For comparison, some subtilisin proteases outside the invention were also included, viz. from Bacillus halmapalus NCIB 12513 (described in WO 88/01293 and also in WO 98/012005 (SEQ ID NO: 42, Bacillus sp. JP170)), and from Bacillus sp. NCIMB 40339 (described in WO 92/017577 as Bacillus sp. TY145). These proteases all have a percentage identity to amino acids 1-274 of SEQ ID NO: 1 of below 50%. Furthermore, the non-subtilisin Nocardiopsis protease described in WO 2005/115445 (amino acids 1-188 of SEQ ID NO: 1 therein) was included for comparison. This protease also has below 50% identity to amino acids 1-274 of SEQ ID NO: 1. Finally, pancreatin was included as a positive control.

The proteases were all dosed equal on an enzyme protein basis, viz. 72, 36, 18, and 9 mg Enzyme Protein (EP) per meal of 250 g. The amount of protease enzyme protein was calculated on the basis of the A₂₈₀ values and the amino acid sequences (amino acid compositions) using the principles outlined in S. C. Gill & P. H. von Hippel, Analytical Biochemistry 182, 319-326, (1989).

### Materials and Methods

Bile salts (i.e. Sodium taurocholate BRP, lot 2, from the Ph.Eur or FIP, also commercially available from e.g. LGC promochem, 500 g/mol), Pepsin (Merck, VL 317492 437 (1.07192)), Pancreatin (from Solvay Pharmaceuticals). Protease diet: 51.9% starch, 21.3% protein and 2.6% fats/lipids.

### In Vitro Model

Protease diet was dissolved in 0.1M HCl to a concentration of 0.2 g diet/mL. pH was adjusted to reach pH 3.0 (simulating gastric conditions). 100 μL diet slurry, 20 μL pepsin (final concentration 70 mg/L in demineralised water (Milli-Q) and 30 μL protease (or Milli-Q in the no-enzyme-control) were added to each well in a microtiter plate (MTP). This was incubated for 1 h at 37° C., 700 rpm. At the end of the 1 hour incubation, pH was measured to 3.4. To raise pH to 6.0 (simulating intestinal conditions), 25 μL of a mixed pH 5/9 buffer (0.8M MES, 0.8M imidazole, 0.8M Na-acetate, pH 5.0 or pH 9.0; 40% pH 5 and 60% pH 9 buffer) were added to each well. Additionally, 25 μL of bile salts (final concentration of 5 mM) was added, and this was incubated 2 h at 37° C., 700 rpm. After in vitro incubation, the MTP's were centrifuged at 2700 rpm (1500 g), 4° C. for 10 min and the supernatants were collected for further investigations.

### Determination of Free Amino Groups (OPA)

The supernatants of the in vitro digestions were analysed by determination of free amino groups by reaction with OPA (O-phthaldialdehyde). The procedure of the OPA determination was as follows; 20 μL diluted in vitro supernatant was transferred to new MTP and added 200 μL OPA reagent (80 mg OPA is dissolved in 2 mL 96% ethanol; 3.81 g di-sodium tetraborate decahydrate, 1 mL 10% SDS, 88 mg DTT and the OPA-ethanol solution is made up to 100 mL with Milli-Q water). Absorbance was measured at 340 nm. A serine standard row (0.5 mg/mL-0.0078 mg/mL) was included in the determinations.

Table 2 below shows the results as mM amino groups hydrolysed. The results are average values of duplicate determinations, and the standard deviation (s.d.) is also indicated. Only the results with 72 mg enzyme protein per meal are shown, as in this test the results with lower enzyme dosages did not allow proper discrimination between the enzymes.

TABLE 2

| Protease tested | SEQ 1 | SEQ 10 | JP170 | TY145 | Nocardiopsis | Pancreatin |
|---|---|---|---|---|---|---|
| Hydrolysed amino groups(mM) | 7.4 | 4.9 | 0.81 | 0.38 | 9.1 | 2.5 |
| S.d. | 0.6 | 2.4 | 0.11 | 0.37 | 1.7 | 1.6 |
| % Identity to SEQ 1 | 100 | 70 | 35 | 47 | 18 | — |

The results of Table 2 show that the proteases of the invention (SEQ 1, SEQ 10) perform very well in this in vitro model. This is not the case for proteases JP170 and TY145 which are not part of the present invention. In fact, disregarding the Nocardiopsis protease which is a quite different type of protease and not included in the present invention, there appears to be a correlation between percentage identities to SEQ ID NO: 1 of the invention and performance in this model (the higher the % identity, the better the performance).

In a separate experiment, performed as described above, we tested the in vitro performance of the Bacillus lentus protease variant of the invention (SEQ 11 variant), including also here for comparison the Nocardiopsis protease. The dose-response results are shown in Table 3 below.

TABLE 3

| Hydrolysed amino | | | | |
|---|---|---|---|---|
| groups (mM) | Protease tested | | S.d. | |
| Enzyme dosage | | SEQ 11 | | SEQ 11 |
| (mg EP/meal) | Nocardiopsis | variant | Nocardiopsis | variant |
| 72 | 6.9 | 3.8 | 0.0 | 0.2 |
| 36 | 5.3 | 3.2 | 0.8 | 0.4 |
| 18 | 4.0 | 2.3 | 0.3 | 0.3 |
| 9 | 2.8 | 1.8 | 0.4 | 0.5 |
| % Identity to SEQ 1 | 18 | 61 | — | — |

Firstly, these results show a good dose-response relationship. Secondly, it is to be noted that also the protease of the invention (SEQ 11 variant) performs very well, in particular at the dosage of 72 mg EP/meal. The SEQ 11 variant even appears to fit well into the correlation between percentage identity to SEQ ID NO: 1 and performance referred to above (relative to the Nocardiopsis protease, which was included in both experiments).

### Example 5

### Pharmaceutical Protease Compositions

### (A) High-Strength Pellets

A germ-filtered liquid concentrate of the protease of amino acids 1-274 of SEQ ID NO: 1 was prepared as described in Example 1 and spray-dried. The measured protease protein content of the spray-dried protease powder was 58.5%. 1125 g spray dried protease in powder form was dry pre-mixed together with microcrystalline cellulose (450 g) and polyethylene glycol 4000 (Macrogol™ 4000; 675 g) in a commercially available mixer. Isopropyl alcohol (460 g; 100%) was added and the resulting wet mass was continued to be thoroughly mixed at room temperature. The homogenized mass was then extruded in a commercially available extruder which was fitted with a piercing die having a hole diameter of 0.8 mm to form cylindrical pellets. The bead temperature was not exceeding 50° C. while extruding. The extrudate produced was rounded to spherical pellets with a commercially available spheronizer by adding the necessary amount of isopropyl alcohol 100% (54.5 g). The pellets were dried at a product temperature of approximately 40° C. in a commercially available vacuum dryer (from Voetsch). The product temperature did not exceed 45° C. The dried pellets were then separated by using a mechanical sieving machine with 0.7 and 1.4 mm screens. The sieve fractions of ≧0.7 mm and ≦1.4 mm were collected and filled in portions of 200 mg pellets each in capsules of size 2. The protease concentration of the resulting dry pellets was approximately 29.3% (w/w).

### (B) Lower-Strength Pellets

Similar to the example provided above (A), pellets with a lower content of protease as drug substance were produced with a batch size of 2250 g using 562.5 g spray dried protease in powder form (with a measured protease protein content of 58.5%), microcrystalline cellulose (1125 g), polyethylene glycol 4000 (562.5 g), isopropyl alcohol for moistening (700 g) and isopropyl alcohol for rounding (61.2 g). The protease concentration of the resulting dry pellets was approximately 14.6% (w/w).

The resulting pellets from examples (A) and (B) were tested for proteolytic activity by applying the FIP method for proteases from pancreas powder with the modification that the activation step was omitted. No loss in proteolytic activity was found in the pellets in each case relative to the starting powdery protease material.

The resulting pellets from examples (A) and (B) were then tested for disintegration according to Pharm. Eur. 2.9.1. (Section “Disintegration of tablets and capsules”) (test solution:water—500 mL, 37° C.).

The disintegration of the pellets from example (A) was completed within 3 min. The disintegration of the pellets from example (B) was completed within 11 min.

### Example 6

### Pharmaceutical Compositions of Protease and Amylase

High-strength pellets containing amylase and protease were prepared as follows:

A liquid concentrate was prepared as described in DK 2005 00931 (a germ-filtered ultrafiltrate) of the amylase having amino acids 1-486 of SEQ ID NO: 16. The liquid concentrate was spray-dried. The measured amylase protein content of the spray-dried amylase powder was 37%. Spray-dried amylase in powder form (398.5 g) was dry pre-mixed together with spray-dried protease powder prepared as described in Example 5 (746.5 g; having a measured protease protein content of 58.5%), microcrystalline cellulose (458 g) and polyethylene glycol 4000 (Macrogol™ 4000; 687 g) in a commercially available mixer. Isopropyl alcohol 100% (460 g) was added and the resulting wet mass was continued to be thoroughly mixed at room temperature. The homogenized mass was then extruded in a commercially available extruder which was fitted with a piercing die having a hole diameter of 0.8 mm to form cylindrical pellets. The bead temperature was not exceeding 50° C. while extruding. The extrudate produced was rounded to spherical pellets with a commercially available spheronizer by adding the necessary amount of isopropyl alcohol 100% (58 g). The pellets were dried using a supply temperature of approximately 40° C. in a commercially available vacuum dryer (from Voetsch). The product temperature did not exceed 45° C. The dried pellets were then separated by using a mechanical sieving machine with 0.7 and 1.4 mm screens. The sieve fractions of ≧0.7 mm and ≦1.4 mm were collected and can be filled in portions of 200 mg each in capsules of size 2. The protease concentration of the resulting dry pellets was approximately 19.1% (w/w), and the amylase concentration of the resulting dry pellets was approximately 6.4% (w/w).

The resulting pellets from were tested for proteolytic and amylolytic activities according to the methods as outlined above. No loss in proteolytic or amylolytic activity was found in the pellets in each case relative to the starting powdery protease or amylase material, respectively.

### Example 7

### Stability and Efficacy In Vivo of Lipase in the Presence of Protease

The stability and efficacy of a Humicola lanuginosa lipase variant of SEQ ID NO: 15 in the presence of a protease of the invention (the protease having amino acids 1-274 of SEQ ID NO: 1) were tested as follows:

The purified lipase was tested in an in vivo trial as generally described in Example 2 of the PCT-application claiming priority from DK application no. 2005 00929, except that dosage was according to lipase units estimated in the pancreatic FIP assay also described in this reference. Digestibility values (coefficient of fat absorption; CFA) were estimated as also described in the referenced patent application.

The lipase was tested alone, and in combination with the protease, in various dosage combinations. The protease activity was determined by using the pancreatic FIP assay (see reference in Example 1).

The results are shown in Table 4 below, given as average CFA (%) values and with indication of the standard deviation (sd).

TABLE 4

| | | Protease | | |
|---|---|---|---|---|
| | | dosage | | |
| | Lipase dosage | (Pancreatic | | |
| | (Pancreatic FIP | FIP | CFA | |
| Treatment | Units per meal) | Units per meal) | (%) | sd |
| Untreated PEI (Control) | 0 | 0 | 21.7 | 4.5 |
| Lipase alone | 107200 | 0 | 59.2 | 4.7 |
| Lipase + Protease | 107200 | 1200 | 55.6 | 6.7 |
| Lipase + Protease | 107200 | 2400 | 58.7 | 5.1 |
| Lipase alone | 780892 | 0 | 75.6 | 4.7 |
| Lipase + Protease | 780892 | 9000 | 81.4 | 4.0 |
| Lipase + Protease | 780892 | 18000 | 76.0 | 3.2 |

For each of the two lipase dosages tested there was no significant difference between the results without and with protease, in the two different dosages. It can therefore be concluded that the protease had no adverse effect on the lipase in vivo.

1-19. (canceled)

20. An isolated protease having at least 50% identity to amino acids 1-274 of SEQ ID NO: 2, for use as a medicament.

21. The protease of claim 20, wherein
(a) the protease comprises an amino acid sequence selected from the group consisting of amino acids 1-274 of SEQ ID NO: 2, amino acids 1-274 of SEQ ID NO: 5, amino acids 1-275 of SEQ ID NO: 6, amino acids 1-275 of SEQ ID NO: 7, amino acids 1-275 of SEQ ID NO: 8, amino acids 1-275 of SEQ ID NO: 9, amino acids 1-275 of SEQ ID NO: 10, amino acids 1-269 of SEQ ID NO: 11, amino acids 1-269 of SEQ ID NO: 12, and amino acids 1-268 of SEQ ID NO: 13; and/or
(b) the protease is a variant of an amino acid sequence selected from the group consisting of amino acids 1-274 of SEQ ID NO: 2, amino acids 1-274 of SEQ ID NO: 5, amino acids 1-275 of SEQ ID NO: 6, amino acids 1-275 of SEQ ID NO: 7, amino acids 1-275 of SEQ ID NO: 8, amino acids 1-275 of SEQ ID NO: 9, amino acids 1-275 of SEQ ID NO: 10, amino acids 1-269 of SEQ ID NO: 11, amino acids 1-269 of SEQ ID NO: 12, and amino acids 1-268 of SEQ ID NO: 13, wherein the variant differs from the respective amino acid sequence by no more than twenty-five amino acids, and wherein:
(i) the variant comprises at least one substitution, deletion and/or insertion of one or more amino acids as compared to the respective amino acid sequence; and/or
(ii) the variant comprises at least one small deletion as compared to the respective amino acid sequence; and/or
(iii) the variant comprises at least one small N- or C-terminal extension as compared to the respective amino acid sequence; and/or
(c) the protease is an allelic variant of a protease having amino acids selected from the group consisting of amino acids 1-274 of SEQ ID NO: 2, amino acids 1-274 of SEQ ID NO: 5, amino acids 1-275 of SEQ ID NO: 6, amino acids 1-275 of SEQ ID NO: 7, amino acids 1-275 of SEQ ID NO: 8, amino acids 1-275 of SEQ ID NO: 9, amino acids 1-275 of SEQ ID NO: 10, amino acids 1-269 of SEQ ID NO: 11, amino acids 1-269 of SEQ ID NO: 12, and amino acids 1-268 of SEQ ID NO: 13; and/or
(d) the protease is a fragment of a protease having amino acids selected from the group consisting of amino acids 1-274 of SEQ ID NO: 2, amino acids 1-274 of SEQ ID NO: 5, amino acids 1-275 of SEQ ID NO: 6, amino acids 1-275 of SEQ ID NO: 7, amino acids 1-275 of SEQ ID NO: 8, amino acids 1-275 of SEQ ID NO: 9, amino acids 1-275 of SEQ ID NO: 10, amino acids 1-269 of SEQ ID NO: 11, amino acids 1-269 of SEQ ID NO: 12, and amino acids 1-268 of SEQ ID NO: 13.

22. The protease of claim 21, wherein the protease has an amino acid sequence selected from the group consisting of amino acids 1-274 of SEQ ID NO: 2, amino acids 1-274 of SEQ ID NO: 5, amino acids 1-275 of SEQ ID NO: 6, amino acids 1-275 of SEQ ID NO: 7, amino acids 1-275 of SEQ ID NO: 8, amino acids 1-275 of SEQ ID NO: 9, amino acids 1-275 of SEQ ID NO: 10, amino acids 1-269 of SEQ ID NO: 11, amino acids 1-269 of SEQ ID NO: 12, and amino acids 1-268 of SEQ ID NO: 13.

23. The protease of claim 22, wherein the protease has amino acids 1-274 of SEQ ID NO: 2.

24. A pharmaceutical composition comprising a protease of claim 20 and at least one pharmaceutically acceptable auxiliary material.

25. The composition of claim 24, further comprising an amylase.

26. The composition of claim 25, wherein the amylase has at least 70% identity to an amylase selected from the group consisting of:
(a) an amylase having amino acids 1-481 of SEQ ID NO: 16,
(b) an amylase having amino acids 1-481 of SEQ ID NO: 17, and
(c) an amylase having amino acids 1-483 of SEQ ID NO: 18.

27. The composition of claim 24, further comprising a lipase.

28. The composition of claim 27, wherein the lipase has at least 70% identity to a lipase having amino acids 1-269 of SEQ ID NO: 15

29. The composition of claim 24, further comprising a lipase and an amylase.

30. The composition of claim 29, wherein
(a) the lipase has at least 70% identity to a lipase having amino acids 1-269 of SEQ ID NO: 15; and
(b) the amylase has at least 70% identity to an amylase selected from the group consisting of:
(i) an amylase having amino acids 1-481 of SEQ ID NO: 16,
(ii) an amylase having amino acids 1-481 of SEQ ID NO: 17, and
(iii) an amylase having amino acids 1-483 of SEQ ID NO: 18.

31. The composition of claim 29, wherein
(a) the protease has amino acids 1-274 of SEQ ID NO: 2;
(b) the lipase comprises amino acids 2-269 of SEQ ID NO: 15; and
(c) the amylase is an amylase selected from the group consisting of:
(i) an amylase comprising amino acids 1-481 of SEQ ID NO: 16,
(ii) an amylase having amino acids 1-481 of SEQ ID NO: 17, and
(iii) an amylase having amino acids 1-483 of SEQ ID NO: 18.

32. A method for the treatment of digestive disorders, pancreatic exocrine insufficiency, pancreatitis, cystic fibrosis, diabetes type I, and/or diabetes type II, comprising administering a therapeutically effective amount of a protease of claim 20.

33. The method of claim 32, further comprising administering a therapeutically effective amount of an amylase.

34. The method of claim 32, further comprising administering a therapeutically effective amount of a lipase.

35. The method of claim 32, further comprising administering a therapeutically effective amount of a lipase and an amylase.
